(19) United States
(12) Patent Application Publication
(10) Pub. No.: US 2024/0392371 A1
(43) Pub. Date: Nov. 28, 2024

(54) STROKE POLYGENIC RISK SCORE AND PATHOGENESIS RISK EVALUATION DEVICE AND APPLICATION THEREOF

(71) Applicant: Fuwai Hospital, Chinese Academy of Medical Sciences and Peking Union Medical College, Beijing City (CN)

(72) Inventors: Xiangfeng Lu, Beijing City (CN); Dongfeng Gu, Beijing City (CN); Jianfeng Huang, Beijing City (CN); Hongfan Li, Beijing City (CN); Xiaoge Niu, Beijing City (CN)

(21) Appl. No.: 18/548,139

(22) PCT Filed: Feb. 28, 2022
(86) PCT No.: PCT/CN2022/078254

§ 371 Date: Aug. 2, 2024

(30) Foreign Application Priority Data
Feb. 26, 2021 (CN) 202110215682.8

(51) Int. Cl. C12Q 1/6883; G16B 20/20; G16H 50/30

(52) U.S. Cl. CPC C12Q 1/6883; G16B 20/20; G16H 50/30; C12Q 2600/118; C12Q 2600/156

(57) ABSTRACT

Provided are a stroke polygenic risk score (PRS) and a pathogenesis risk evaluation device and an application thereof. Specifically, provided is an application of a reagent, which is used for detecting individual information, in preparation of a detection device for evaluating a pathogenesis risk of stroke, wherein the individual information comprises 280 Stroke-related single nucleotide polymorphism sites. The individual information preferably further comprises one or more of CAD, SBP, WC, T2D, TC, PP, and AF-related single nucleotide polymorphism sites. The PRS and a traditional risk factor are further integrated, so that re-stratification of the pathogenesis risk of stroke can be achieved, and important significance for primary prevention of stroke is achieved.

### TECHNICAL FIELD

The present invention relates to a polygenic risk score (PRS) for stroke and an incidence risk evaluation device and applications thereof.

### BACKGROUND

Death from stroke is one of the major global health threats. The lifetime risk of stroke in adults over age 25 is estimated to be about 25% globally, with East Asian populations having the highest risk of up to 39%. In China, stroke is the leading cause of death among the population, with 2.07 million stroke deaths in 2017. Therefore, early identification of high-risk groups, healthy lifestyle management and pharmacological intervention for major risk factors (e.g. hypertension, diabetes, dyslipidaemia, etc.) are important for primary prevention of stroke in China and in the world.

Stroke is a complex disease caused by a combination of genetic and environmental factors. Genome-wide association studies (GWAS) have identified at least 35 genetic susceptibility genes associated with stroke and hundreds of genes associated with stroke-related phenotypes including blood pressure, type 2 diabetes (T2D), lipid levels, body mass index (BMI), and atrial fibrillation (AF). The identification of these genetic variants will help to develop cardiovascular disease risk prediction and guide primary prevention. Recently, a polygenic risk score (PRS) for stroke, which integrates information from multiple genetic variants, has been successfully developed and applied to the clinical evaluation of stroke risk prediction.

However, almost all available genetic scores have been constructed based on European populations (Stroke 2014; 45:394-402, Stroke 2014; 45:403-412, Stroke 2014; 45:2856-2862, BMJ 2018; 363: k4168, JAMA cardiology 2018; 3:693-702, Nat Commun 2019; 10:5819), with few reports on those outside the Europe populations. The epidemiological characteristics of stroke vary from country to country, and in East Asian populations, especially in Chinese populations, there is a much higher incidence of stroke and rate of haemorrhagic stroke events compared with Western populations. Therefore, it is crucial to construct a PRS for stroke in East Asian populations, especially in Chinese populations, and to strictly assess its predictive value for genetic risk in a prospective cohort population.

In addition, significant differences in environmental risk factors (lifestyle, diet and behaviour) as well as gene-environment interactions in different populations may also contribute to differential stroke risks and intervention benefits.

In addition, the ability to re-stratify the risk of stroke incidence by integrating polygenic risk scores and traditional risk factors is important for primary prevention of stroke.

### SUMMARY OF THE INVENTION

It is an object of the present invention to provide stroke-associated single nucleotide polymorphism sites and a system for evaluating the risk of stroke incidence applicable to an East Asian population.

The inventors of present application have identified a group of stroke risk-related genes associated with East Asian populations through extensive research and practical detection and analysis tests, which include 280 stroke-associated single nucleotide polymorphism (SNP) sites, and by detecting these SNP sites, the risk of stroke incidence can be well evaluated in East Asian populations. The present invention further identifies CAD, SBP, WC, T2D, TC, PP, and AF-related single nucleotide polymorphism sites, and by further detecting these related single nucleotide polymorphism sites, the risk of stroke incidence in East Asian populations can be better evaluated.

Specifically, in one aspect, the present invention provides the use of a reagent for detecting individual information in the preparation of a detection device for evaluating a risk of stroke incidence, wherein the individual information comprises the following single nucleotide polymorphism site information:
stroke-related single nucleotide polymorphism sites: rs10051787, rs10093110, rs10139550, rs10160804, rs10237377, rs10260816, rs10267593, rs10278336, rs1037814, rs10507248, rs10512861, rs10745332, rs10757274, rs10773003, rs10824026, rs10857147, rs10953541, rs10968576, rs11099493, rs1116357, rs11206510, rs11222084, rs11257655, rs11509880, rs1152591, rs11557092, rs11601507, rs11604680, rs11624704, rs11677932, rs1173766, rs117601636, rs117711462, rs11787792, rs11810571, rs11838776, rs11869286, rs12027135, rs12037987, rs12202017, rs12229654, rs12415501, rs12438008, rs12445022, rs12500824, rs1250229, rs12549902, rs12571751, rs12581963, rs12692735, rs12718465, rs12801636, rs12897, rs12927205, rs12932445, rs12936587, rs12946454, rs13143308, rs13209747, rs1321309, rs13216675, rs13233731, rs13342232, rs1334576, rs13359291, rs1344653, rs1359790, rs1367117, rs13723, rs1412444, rs1436953, rs1470579, rs1495741, rs1508798, rs151193009, rs1552224, rs1591805, rs16844401, rs16849225, rs16858082, rs16896398, rs16967013, rs16999793, rs17030613, rs17080091, rs17087335, rs17122278, rs17135399, rs17301514, rs173396, rs17358402, rs17477177, rs17514846, rs17581137, rs17612742, rs17680741, rs17791513, rs180327, rs181359, rs1861411, rs1868673, rs1870634, rs1887320, rs1892094, rs1902859, rs191835914, rs1976041, rs1982963, rs2000813, rs2028299, rs2057291, rs2068888, rs2074158, rs2075291, rs2075423, rs2107595, rs2128739, rs2145598, rs216172, rs2213732, rs2229383, rs2237896, rs2240736, rs2245019, rs2261181, rs2295786, rs2334499, rs243019, rs246600, rs247616, rs2487928, rs2535633, rs2575876, rs261967, rs273909, rs2758607, rs2782980, rs2796441, rs2815752, rs2820315, rs2861568, rs2925979, rs2972146, rs29941, rs326214, rs340874, rs351855, rs35337492, rs35444, rs36096196, rs368123, rs376563, rs3775058, rs3785100, rs3791679, rs3861086, rs3887137, rs3903239, rs3936511, rs4275659, rs4400058, rs4409766, rs4458523, rs4468572, rs4593108, rs46522, rs4719841, rs4722766, rs4724806, rs4731420, rs4752700, rs4766228, rs4788102, rs4812829, rs4821382, rs4836831, rs4846049, rs4883263, rs4911495, rs4918072, rs4932370, rs556621, rs56062135, rs574367, rs579459, rs582384, rs5996074, rs6093446, rs61776719, rs633185, rs6490029, rs6545814, rs663129, rs6666258, rs667920, rs6700559, rs671, rs67156297, rs67180937, rs6725887, rs67839313, rs6795735, rs6813195, rs6817105, rs6825454, rs6825911, rs6829822, rs6831256, rs6838973, rs6878122, rs6882076, rs6905288, rs6909752, rs6960043, rs699, rs6997340, rs702485, rs702634, rs7136259, rs7164883, rs7178572, rs7193343, rs7199941, rs7202877, rs7206541, rs7258189, rs7258445, rs7258950, rs72689147, rs73015714, rs7304841, rs7306455, rs73069940, rs736699, rs737337, rs7403531, rs740406, rs7499892, rs7500448, rs7503807, rs7568458, rs7610618, rs7616006, rs7696431, rs7770628, rs780094, rs7810507, rs7859727, rs7917772, rs79223353, rs7947761, rs7955901, rs7965082, rs7980458, rs8042271, rs8108269, rs838880, rs840616, rs871606, rs880315, rs884366, rs885150, rs888789, rs9266359, rs9268402, rs9299, rs9319428, rs9376090, rs9473924, rs9505118, rs9568867, rs964184, rs9687065, rs975722, rs9810888, rs9815354, rs9828933, rs984222, rs9892152, rs9970807.

According to a specific embodiment of the present invention, in the present invention, the individual information preferably further comprises one or more of CAD, SBP, WC, and T2D-associated single nucleotide polymorphism sites:
CAD-related single nucleotide polymorphism sites: rs10096633, rs10203174, rs1027087, rs1029420, rs10401969, rs10455782, rs10513801, rs1077834, rs10820405, rs10830963, rs10842992, rs10886471, rs11030104, rs11057830, rs11066280, rs11067763, rs11077501, rs11125936, rs11136341, rs11142387, rs11205760, rs1129555, rs11556924, rs11634397, rs1169288, rs11830157, rs11838267, rs11847697, rs1211166, rs12204590, rs12214416, rs12242953, rs12453914, rs12463617, rs12524865, rs12535846, rs12597579, rs12679556, rs12740374, rs12970066, rs12999907, rs130071, rs13041126, rs13078807, rs1317507, rs13266634, rs13277801, rs13306194, rs1378942, rs1467605, rs1496653, rs1514175, rs1535500, rs1555543, rs1558902, rs1575972, rs1689800, rs16933812, rs16986953, rs16990971, rs17080102, rs17150703, rs17249754, rs17381664, rs174547, rs17465637, rs17517928, rs17609940, rs17678683, rs17695224, rs17843768, rs1799945, rs1800234, rs1801282, rs181360, rs2000999, rs200990725, rs2021783, rs2043085, rs2066714, rs2075260, rs2106261, rs2144300, rs2237892, rs2296172, rs2302593, rs2328223, rs2383208, rs2415317, rs2531995, rs2571445, rs2642442, rs2819348, rs2820443, rs3129853, rs3130501, rs3213545, rs35332062, rs3809128, rs3827066, rs3846663, rs391300, rs3993105, rs4148008, rs4266144, rs4377290, rs439401, rs4420638, rs4471613, rs459193, rs4613862, rs4713766, rs4735692, rs4757391, rs4845625, rs4917014, rs4923678, rs499974, rs5215, rs55783344, rs56289821, rs56336142, rs590121, rs6065311, rs6494488, rs651821, rs660599, rs6807945, rs6808574, rs6818397, rs7087591, rs7107784, rs7116641, rs7225581, rs72654473, rs748431, rs7525649, rs7617773, rs78169666, rs7901016, rs7989336, rs8030379, rs8090011, rs820430, rs867186, rs896854, rs897057, rs9309245, rs93138, rs9349379, rs9357121, rs9367716, rs9390698, rs944172, rs9470794, rs9534262, rs9552911, rs9593, rs995000;
SBP-related single nucleotide polymorphism sites: rs1275988, rs7701094, rs7405452, rs751984;
WC-related single nucleotide polymorphism site: rs2303790;
T2D-related single nucleotide polymorphism sites: rs10010670, rs10064156, rs1052053, rs10923931, rs11651052, rs11660468, rs1260326, rs13143871, rs1448818, rs1532085, rs16927668, rs174546, rs17608766, rs17843797, rs1800588, rs1832007, rs2081687, rs2123536, rs2156552, rs2230808, rs2258287, rs2297991, rs2783963, rs2954029, rs3807989, rs3810291, rs3918226, rs4142995, rs42039, rs4302748, rs4776970, rs4883201, rs58542926, rs60154123, rs6038557, rs634501, rs6871667, rs6984210, rs7185272, rs7208487, rs7213603, rs738409, rs7528419, rs7678555, rs769449, rs76954792, rs7897379, rs7903146, rs79548680, rs80234489, rs806215, rs9501744, rs9512699, rs9591012, rs9818870.

According to a specific embodiment of the present invention, in the present invention, the individual information more preferably further comprises one or more of TC, PP, and AF-related single nucleotide polymorphism sites:
TC-related single nucleotide polymorphism sites: rs10889353, rs11957829, rs13115759, rs1421085, rs1424233, rs1805081, rs1883025, rs2625967, rs2972143, rs3120140, rs3184504, rs34008534, rs4129767, rs4939883, rs507666, rs515135, rs6544713, rs7134594, rs7306523, rs7560163, rs7633770, rs9663362;
PP-related single nucleotide polymorphism sites: rs10821415, rs11196288, rs312949, rs1333042, rs1867624, rs2292318, rs2519093, rs35419456, rs7916879;
AF-related single nucleotide polymorphism sites: rs11191416, rs1200159, rs12042319, rs2200733.

According to a specific embodiment of the present invention, in the present invention, the individual information preferably further comprises clinical factors comprising the presence or absence of a stroke family history, hypertension, diabetes, dyslipidaemia and/or obesity.

According to a specific embodiment of the present invention, in the present invention, a genetic risk score is obtained based on the information of respective single nucleotide polymorphism sites in accordance with the following calculation:

Genetic risk score=Σβi×Ni where Bi is the effect value of the i^{th} SNP and Ni is the number of effect alleles of the i^{th} SNP carried by the individual.

According to a specific embodiment of the present invention, in the present invention, the effect values of each SNP are shown in Table 3.

According to a specific embodiment of the present invention, in the present invention, the higher the genetic risk score, the higher the risk of stroke incidence in the individual. Said stroke comprises haemorrhagic stroke and/or ischaemic stroke.

According to a specific embodiment of the present invention, in the present invention, the individual to be evaluated is from an East Asian population, especially Chinese.

In another aspect, the present invention also provides a device for evaluating a risk of stroke incidence comprising a detection unit and a data analysis unit, wherein:
the detection unit is used for detecting information from an individual to be evaluated and obtaining detection results; wherein the individual information is the individual information described as defined in any one of claims 1 to 3;
the data analysis unit is used for analyzing and processing the detection results from the detection unit.

According to a specific embodiment of the present invention, in the present invention, the analyzing and processing the detection results from the detection unit by the data analysis unit comprises: assigning a weight factor to the detection result of the single nucleotide polymorphism sites to calculate a genetic risk score of the individual to be evaluated;
preferably, the data analysis unit comprises:
a preprocessing module for normalizing the detection results of the single nucleotide polymorphism sites;
a calculation module for bringing the normalized detection results of the single nucleotide polymorphism sites into following evaluation model to obtain a genetic risk score for the individual to be evaluated:

Genetic risk score=Σβi×Ni
where βi is the effect value of the i^{th} SNP and Ni is the number of effect alleles of the i^{th} SNP carried by the individual.

According to a specific embodiment of the present invention, in the present invention, the calculation module is used to evaluate lifetime stroke risk information by further combining the genetic risk score with clinical factors.

According to a specific embodiment of the present invention, in the present invention, the data analysis unit further comprises:
a matrix input module for receiving a plurality of the normalized detection results output by the preprocessing module, inputting the normalized detection results in a matrix form to the calculation module;
preferably, the data analysis unit further comprises:
an output module for receiving the genetic risk score and/or the lifetime stroke risk information output by the calculation module and outputting it as a diagnostic classification result.

In another aspect, the present invention also provides a computer storage medium storing computer program instructions, wherein when the computer program instructions are executed, an evaluation result of the risk of stroke incidence in an individual is obtained based on the information about the individual to be evaluated. Here, the individual information is as previously described.

In yet another aspect, the present invention also provides a computer device comprising a memory, a processor and a computer program that is stored in the memory and executable on the processor, wherein when the processor executes the computer program, an evaluation result of the risk of stroke incidence in an individual is obtained based on information about the individual to be evaluated. Here, the individual information is as previously described.

In a specific embodiment of the present invention, the present invention relies on a Chinese large prospective cohort population to identify stroke risk-related single nucleotide polymorphism sites associated with East Asian populations, develops a polygenic risk score that includes multiple genetic variants, and evaluates its effect on stroke risk stratification in a large prospective cohort of 41,006 study subjects, alone or in combination with traditional risk factors (hypertension, diabetes, dyslipidaemia, obesity, and family history of stroke). The study has found that individuals with a high genetic risk (the upper 20% at a genetic risk) had an approximately 2-fold higher risk of stroke (HR: 1.99, 95% CI: 1.66-2.38) than those with a low genetic risk (the lower 20% at a genetic risk), and the lifetime risk of stroke in the two groups was 25.2% (95% CI: 22.5%-27.7%) and 13.6% (95% CI: 11.6%-15.5%). There was a significant difference in the stroke profile between the groups by stratification with the genetic risk score in combination with traditional risk factors. Individuals with a low genetic risk and no family history of stroke had a 13.2% lifetime risk of stroke, while those with either one of them had an approximately two fold increased risk of stroke (23.9%, 95% CI: 21.1%-26.5%, and 23.7%, 95% CI: 13.4%-32.8%) and individuals with both a high genetic risk and a family history of stroke had the highest lifetime risk of stroke (41.1%, 95% CI: 31.4%-49.5%). In addition, the risk evaluation for stroke incidence of the present invention is applicable to both haemorrhagic and ischaemic strokes. The present study confirms that a combination of polygenic risk scores and traditional risk factors can lead to a refined re-stratification of stroke risk, e.g., the application of this polygenic risk score allowed early identification of 20% of the general population whose lifetime risk of stroke was comparable to that of those with a family history of stroke. Individual stroke risk further increases when a high genetic risk is combined with a family history of stroke, and may reach 40% or more. In clinical applications, the combination of a genetic risk and a family history may be of key guidance to early screening for stroke. In addition, simultaneous integration of polygenic risk scores with traditional risk factors for hypertension, diabetes, dyslipidemia, and obesity leads to a similar observation of significant differences in stroke profiles among the groups. The above results highlight the merits in application by integrating polygenic risk scores and traditional risk factors to achieve refined re-stratification of stroke incidence risk and to guide early screening and individualised intervention in high-risk populations. The present invention has great prospects of application in the primary prevention of stroke.

### DESCRIPTION OF THE DRAWINGS

FIG. 1 shows the association of candidate polygenic risk scores (per standard deviation increment) with stroke in a training set.

FIG. 2 shows the association of the best polygenic risk score (per standard deviation increment) with stroke in a training set.

FIG. 3 shows the correlation between metaPRS and the best subphenotypic polygenic risk score in a prospective cohort.

FIG. 4 shows the association of metaPRS and the best subphenotypic polygenic risk score with stroke incidence in a prospective cohort.

FIG. 5 shows the lifetime risk of stroke for different genetic risks.

FIG. 6 shows the lifetime risk of stroke for different genetic and stroke family history stratifications.

FIG. 7 shows the association of metaPRS quintiles with stroke incidence.

FIG. 8 shows the lifetime risk of stroke for different genetic and clinical risk factor subgroups.

FIG. 9 shows the lifetime risk of ischaemic and haemorrhagic stroke stratified by different genetic risks. FIG. 10 shows the lifetime risk of ischaemic and haemorrhagic stroke stratified by different genetic and major risk factors. Hazard ratios (HR) and cumulative incidence curves for ischaemic and haemorrhagic strokes before age 80, adjusted for sex, are calculated using Cox proportional risk regression models with cohort stratification and age as the time scale in FIGS. 9 and 10.

### DETAILED DESCRIPTION OF THE INVENTION

In order to have a better understanding of the technical features, purposes and beneficial effects of the present invention, detailed description of the technical solution of the present invention is given in conjunction with specific examples hereinbelow, and it should be understood that these examples are only used to illustrate the present invention and are not intended to limit the scope of the present invention. In the examples, each of the original reagent materials is commercially available, and the experimental methods for which the specific conditions are not indicated are conventional methods and conventional conditions well known in the related art, or as recommended by the instrument manufacturer.

### Example 1

### Research Design Procedure and Study Population

In this study, a metaPRS was constructed using a training set with a case-control design, and its clinical value for stroke risk prediction was validated and evaluated in a large prospective cohort, “Prediction for Atherosclerotic cardiovascular disease Risk in China (China-PAR)”.

The training set consisted of 2872 stroke cases (2548 ischaemic and 324 haemorrhagic strokes) and 2494 controls (Table 1). Stroke cases came from hospitals and were diagnosed by neurologists based on medical records of computed tomography (CT) scans and/or magnetic resonance imaging (MRI). The control group was randomly selected from individuals who participated in the Community Cardiovascular Risk Factor Survey and had not had a stroke as determined by medical history, clinical examination, and standard questionnaires.

The validation population was drawn from three cohorts of the China-PAR project: the China Multi-Center Collaborative Study of Cardiovascular Epidemiology 1998 (China MUCA 1998), the International Collaborative Study of Cardiovascular Disease in Asia (InterASIA), and the Community Intervention of Metabolic Syndrome in China & Chinese Family Health Study (CIMIC). The latest follow-ups of the three cohorts were conducted during 2012-2015 using a uniform questionnaire and protocol. Of the 43,88 1 participants for whom blood samples and follow-up information were available, the present invention further excluded 561 participants with a high genotypic deletion rate (>5.0%) or low mean sequencing depth (<30×), 1,352 participants with a baseline age of <30 or >75 years, and 962 participants with a cardiovascular disease (stroke and myocardial infarction) at baseline, for a total of 41,006 participants eventually included in the analysis.

The studies were approved by the Ethical Review Committee of Fu Wai Hospital, Chinese Academy of Medical Sciences. Each participant had signed a written informed consent before data collection.

TABLE 1
Population characteristics of the training set

| | Control | | Stroke cases | |
|---|---|---|---|---|
| Characteristics | (N = 2494) | | (N = 2872) | |
| Age at the time of participation | 66.1 | (10.3) | — | |
| in the study, years | | | | |
| Age of incidence, years | — | | 66.6 | (9.8) |
| Male, N (%) | 934 | (37.4) | 1,617 | (56.3) |
| Current smoker, N (%) | 554 | (22.2) | 622 | (21.8) |
| Systolic blood pressure, mmHg | 132.4 | (15.9) | 149.7 | (23.7) |
| Diastolic blood pressure, mmHg | 82.9 | (8.5) | 87.9 | (25.9) |
| Total cholesterol, mg/dl | 188.1 | (36.8) | 182.3 | (64.5) |
| Hypertension, N (%) | 1,176 | (47.2) | 2,242 | (78.9) |
| Diabetes, N (%) | 285 | (11.4) | 578 | (20.3) |
| Dyslipidemia, N (%) | 895 | (35.9) | 1,330 | (48.5) |

Continuous variables are expressed as mean (standard deviation) and categorical variables are expressed as number (percentage).

### Collection of Major Traditional Risk Factors at Baseline

In the baseline survey, a standard questionnaire, physical examination and laboratory tests were conducted for each participant. A series of lifestyle risk factors and cardiovascular metabolic indicators were collected by professionally trained and qualified investigators according to a uniformly developed survey protocol. The main traditional risk factors for stroke at baseline include hypertension, dyslipidaemia, diabetes, obesity (BMI ≥28 kg/m²), and family history of stroke. Hypertension was defined by systolic blood pressure (SBP) ≥140 mmHg and/or diastolic blood pressure (DBP)>90 mmHg and/or use of antihypertensive medication within the past two weeks. Dyslipidaemia was defined by total cholesterol (TC) ≥240 mg/d1 and/or high-density lipoprotein cholesterol (HDL-C)<40 mg/d1 and/or triglycerides (TG) ≥200 mg/d1 and/or low-density lipoprotein cholesterol (LDL-C) ≥160 mg/d1 and/or use of lipid-lowering medication. Diabetes was defined by fasting blood glucose >126 mg/d1 and/or use of insulin or oral hypoglycaemic medication. Family history of stroke was defined as a history of stroke in any first-degree relative (father, mother, or siblings).

### Follow-up of Stroke Events

The three cohorts were followed up using the same study protocol, and information on stroke morbidity and mortality was obtained from the study subjects by appointment and household surveys, and medical records and death certificates were further obtained for verification. All medical and death records were independently reviewed by two experts from the Endpoint Evaluation Committee of Fu Wai Hospital, Chinese Academy of Medical Sciences. If the two experts' opinions were not unanimous, discussion was conducted by the other experts on the committee to reach a final diagnosis. Causes of death were coded according to ICD-10 (International Classification of Diseases, the 10^{th} Edition). Stroke was defined as a first fatal or non-fatal stroke event diagnosed during the follow-up (160-169). Stroke subtypes were classified as ischaemic stroke (163), haemorrhagic stroke (160-162) and unspecified subtype of stroke (164-169).

### Selection and Genotyping of Single Nucleotide Polymorphic Sites

The present invention selected 588 single nucleotide polymorphism (SNP) sites that achieve genome-wide significant association with stroke or stroke-related phenotypes based on previous genome-wide association studies (Table 2).

TABLE 2
Number of SNPs selected for the study

| Traits | No. of SNPs |
|---|---|
| Stroke (AS, IS, HS) | 42 |
| BP (SBP, DBP, PP, MAP, hypertension) | 46 |
| CAD | 199 |
| T2D | 89 |
| Obesity (BMI, WC, WHR) | 79 |
| Lipids (TC, LDL-C, TG, HDL-C) | 126 |
| AF | 16 |
| Total | 588* |

*The sum does not add up to 588 due to overlapping susceptible SNPs between phenotypes (equal to 597).
SNP, single nucleotide polymorphism; AS, all strokes; IS, ischaemic stroke; HS, haemorrhagic stroke; BP, blood pressure; SBP, systolic blood pressure; DBP, diastolic blood pressure; PP, pulse pressure; MAP, mean arterial pressure; CAD, coronary artery disease; T2D, type 2 diabetes; BMI, body mass index; WC, waist circumference; WHR, waist-to-hip ratio; TC, total cholesterol; LDL-C, low-density lipoprotein cholesterol; TG, triglyceride; HDL-C, high-density lipoprotein cholesterol; AF, atrial fibrillation.

All participants in the training and validation sets were genotyped using multiplex polymerase chain reaction targeted amplicon sequencing. Target regions were amplified for high-throughput sequencing using an Illumina Hiseq X Ten sequencer. After excluding SNPs with less than 95% genotype detection rate, 578 autosomal SNPs were retained for subsequent analysis, with an average genotype detection rate of 99.9% and a median sequencing depth of 979×. To assess the reproducibility of genotyping, 1648 samples in duplicate were tested and the genotyping concordance rate was >99.4%.

Construction of metaPRS

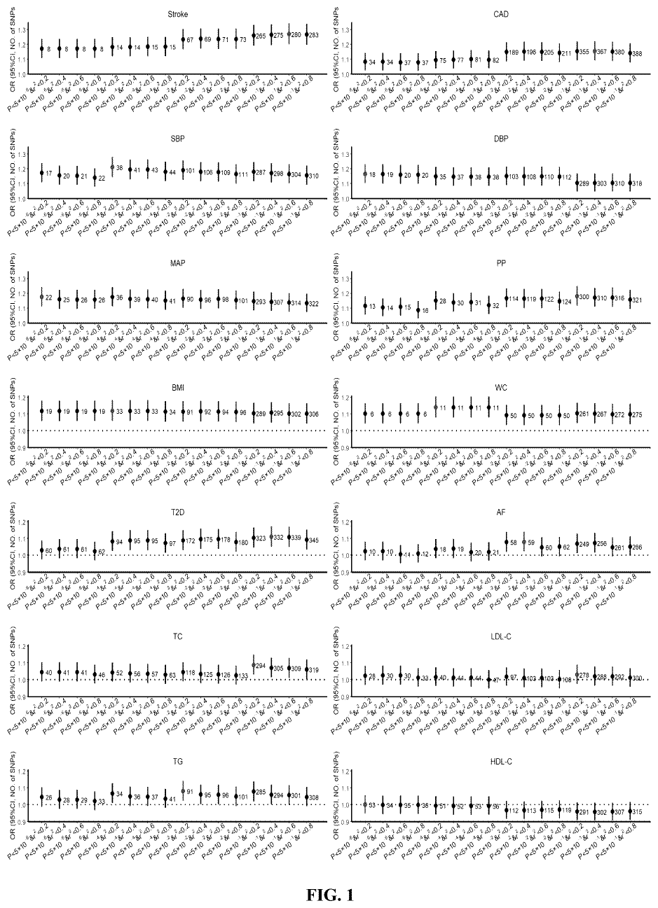

The number of alleles per variant (0, 1, or 2) per individual was weighted and summed according to the effect value of its corresponding allele in that phenotype to construct 14 stroke-related subphenotype-specific PRSs (stroke, coronary heart disease, type 2 diabetes, atrial fibrillation, systolic blood pressure, diastolic blood pressure, mean arterial pressure, pulse pressure, body mass index, waist circumference, total cholesterol, LDL cholesterol, triglycerides, and HDL cholesterol). For each subphenotype, 16 candidate PRSs were constructed based on the pooled data using different linkage disequilibrium r² (0.2, 0.4, 0.6, 0.8) and significance thresholds (P-value=0.5, 0.05, 5×10⁻⁴, 5×10⁻⁶). The association of these candidate PRSs with stroke was evaluated in the training set using a logistic regression model, and the score with the largest odds ratio (OR) (for each standard deviation increment in the PRS) was selected as the best PRS (FIG. 1). Among them, the SNP sites and effect sizes used for the best stroke sub-phenotype (Stroke) PRS are shown in Table 3.

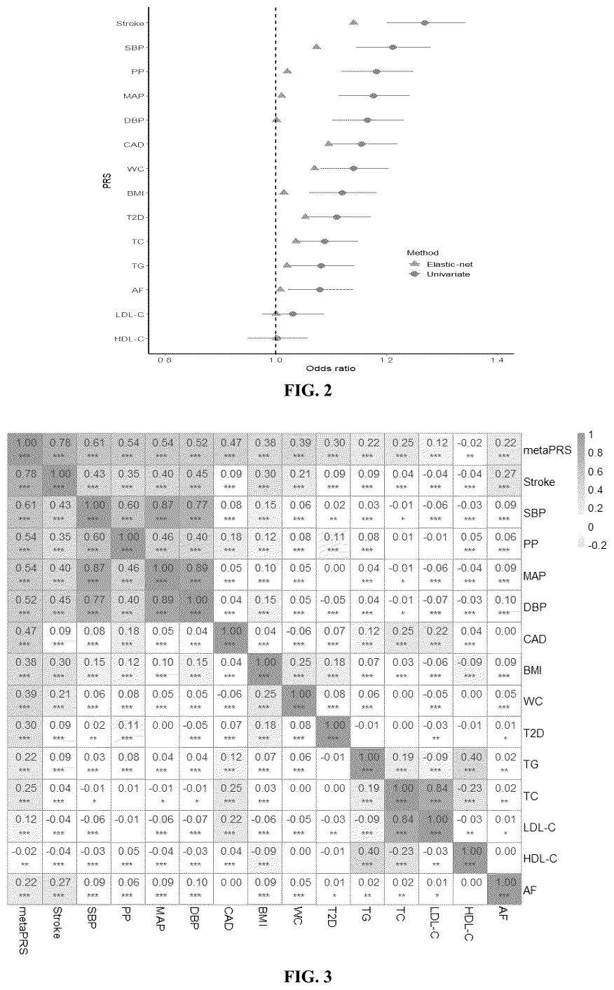

Each of the best PRS was converted into a score with a mean of 0 and a standard deviation of 1. The association between the 14 best PRSs and stroke was modelled using elastic net logistic regression with 10-fold cross-validation (R package “glmnet”) and further constructed as a metaPRS. The model with the highest area under receiving-operator characteristic curve (AUC) was selected as the final model, from which the correction coefficients for each PRS were obtained as weights. The corrected effect values for each PRS by the univariate estimation (based on one PRS at a time) and the elastic net logistic regression estimation are shown in FIG. 2. After the statistical processing procedure, a total of 534 SNPs were finally included in the metaPRS calculation, and the information and weights of all eligible SNPs are provided in Table 3.

TABLE 3
Information and weights of SNPs identified by the present invention

| | Subphenotypic PRS | | | | | MetaPRS | | |
|---|---|---|---|---|---|---|---|---|
| | | risk | other | SNP effect | subphenotypic | effector | other | SNP effect |
| SNP | subphenotype | allele | alleles | value | PRS weight | allele | alleles | value |
| rs10051787 | Stroke | T | C | 0.020727 | 0.130873 | T | C | 0.018973126 |
| rs10093110 | Stroke | G | A | 0.035289 | 0.130873 | A | G | −0.019754829 |
| rs10139550 | Stroke | G | C | 0.025013 | 0.130873 | G | C | 0.016337681 |
| rs10160804 | Stroke | A | C | 0.009121 | 0.130873 | A | C | 0.015460378 |
| rs10237377 | Stroke | G | T | 0.014063 | 0.130873 | G | T | 0.012350791 |
| rs10260816 | Stroke | C | G | 0.011287 | 0.130873 | C | G | 0.012440076 |
| rs10267593 | Stroke | G | A | 0.017825 | 0.130873 | A | G | −0.014634354 |
| rs10278336 | Stroke | A | G | 0.015523 | 0.130873 | G | A | −0.012139344 |
| rs1037814 | Stroke | T | C | 0.015238 | 0.130873 | T | C | 0.006616393 |
| rs10507248 | Stroke | T | G | 0.030201 | 0.130873 | T | G | 0.016667477 |
| rs10512861 | Stroke | G | T | 0.044836 | 0.130873 | T | G | −0.02726483 |
| rs10745332 | Stroke | A | G | 0.023669 | 0.130873 | G | A | −0.015383705 |
| rs10757274 | Stroke | G | A | 0.019385 | 0.130873 | G | A | 0.033064463 |
| rs10773003 | Stroke | A | G | 0.023617 | 0.130873 | A | G | 0.005781325 |
| rs10824026 | Stroke | G | A | 0.012005 | 0.130873 | G | A | 0.008192385 |
| rs10857147 | Stroke | T | A | 0.061049 | 0.130873 | T | A | 0.06863237 |
| rs10953541 | Stroke | C | T | 0.019038 | 0.130873 | T | C | −0.007903768 |
| rs10968576 | Stroke | G | A | 0.013832 | 0.130873 | G | A | 0.011499091 |
| rs11099493 | Stroke | A | G | 0.021201 | 0.130873 | G | A | −0.011677411 |
| rs1116357 | Stroke | G | A | 0.025136 | 0.130873 | G | A | 0.015769568 |
| rs11206510 | Stroke | T | C | 0.023438 | 0.130873 | C | T | −0.02528128 |
| rs11222084 | Stroke | A | T | 0.018241 | 0.130873 | T | A | −0.002905714 |
| rs11257655 | Stroke | C | T | 0.011761 | 0.130873 | C | T | −0.011322812 |
| rs11509880 | Stroke | A | G | 0.011471 | 0.130873 | G | A | −0.008942689 |
| rs1152591 | Stroke | G | A | 0.011717 | 0.130873 | A | G | −0.003268347 |
| rs11557092 | Stroke | C | T | 0.014805 | 0.130873 | T | C | −0.019002183 |
| rs11601507 | Stroke | C | A | 0.025773 | 0.130873 | A | C | −0.004847344 |
| rs11604680 | Stroke | G | A | 0.02846 | 0.130873 | G | A | 0.017626551 |
| rs11624704 | Stroke | C | A | 0.026635 | 0.130873 | C | A | 0.016364282 |
| rs11677932 | Stroke | G | A | 0.017636 | 0.130873 | A | G | −0.004910852 |
| rs1173766 | Stroke | C | T | 0.025602 | 0.130873 | T | C | −0.033919358 |
| rs117601636 | Stroke | A | G | 0.020598 | 0.130873 | G | A | −0.031314005 |
| rs117711462 | Stroke | A | G | 0.175926 | 0.130873 | A | G | 0.098447543 |
| rs11787792 | Stroke | A | G | 0.03053 | 0.130873 | G | A | −0.029985673 |
| rs11810571 | Stroke | G | C | 0.028718 | 0.130873 | G | C | 0.017230874 |
| rs11838776 | Stroke | A | G | 0.045797 | 0.130873 | A | G | 0.025916043 |
| rs11869286 | Stroke | G | C | 0.01526 | 0.130873 | C | G | −0.006136339 |
| rs12027135 | Stroke | T | A | 0.016499 | 0.130873 | T | A | 0.00639276 |
| rs12037987 | Stroke | C | T | 0.051649 | 0.130873 | C | T | 0.054730085 |
| rs12202017 | Stroke | G | A | 0.016382 | 0.130873 | G | A | 0.007572326 |
| rs12229654 | Stroke | T | G | 0.089997 | 0.130873 | G | T | −0.070165899 |
| rs12415501 | Stroke | T | C | 0.031594 | 0.130873 | T | C | 0.013439712 |
| rs12438008 | Stroke | G | A | 0.018336 | 0.130873 | A | G | 0.006656662 |
| rs12445022 | Stroke | A | G | 0.06483 | 0.130873 | A | G | 0.026427837 |
| rs12500824 | Stroke | A | G | 0.008382 | 0.130873 | A | G | 0.011592952 |
| rs1250229 | Stroke | T | C | 0.024033 | 0.130873 | T | C | 0.021836574 |
| rs12549902 | Stroke | G | A | 0.013561 | 0.130873 | A | G | 0.003174603 |
| rs12571751 | Stroke | A | G | 0.019032 | 0.130873 | G | A | −0.012841736 |
| rs12581963 | Stroke | G | A | 0.029512 | 0.130873 | A | G | −0.012560115 |
| rs12692735 | Stroke | G | T | 0.017416 | 0.130873 | T | G | −0.016576062 |
| rs12718465 | Stroke | C | T | 0.036909 | 0.130873 | T | C | −0.019783756 |
| rs12801636 | Stroke | G | A | 0.023377 | 0.130873 | A | G | −0.030230439 |
| rs12897 | Stroke | G | A | 0.018393 | 0.130873 | A | G | −0.017003901 |
| rs12927205 | Stroke | G | A | 0.028147 | 0.130873 | G | A | −0.0026303 |
| rs12932445 | Stroke | C | T | 0.033092 | 0.130873 | C | T | 0.014000779 |
| rs12936587 | Stroke | A | G | 0.030176 | 0.130873 | A | G | 0.009266501 |
| rs12946454 | Stroke | A | T | 0.015626 | 0.130873 | T | A | −0.002921822 |
| rs13143308 | Stroke | T | G | 0.029245 | 0.130873 | G | T | −0.009405478 |
| rs13209747 | Stroke | T | C | 0.02166 | 0.130873 | T | C | 0.019474403 |
| rs1321309 | Stroke | A | G | 0.010475 | 0.130873 | A | G | 0.013167272 |
| rs13216675 | Stroke | T | C | 0.022355 | 0.130873 | C | T | −0.007377649 |
| rs13233731 | Stroke | G | A | 0.011327 | 0.130873 | A | G | −0.01474881 |
| rs13342232 | Stroke | G | A | 0.020812 | 0.130873 | G | A | 0.021047598 |
| rs1334576 | Stroke | A | G | 0.009331 | 0.130873 | G | A | −0.006975739 |
| rs13359291 | Stroke | A | G | 0.023185 | 0.130873 | G | A | −0.007962384 |
| rs1344653 | Stroke | G | A | 0.021965 | 0.130873 | G | A | 0.009267282 |
| rs1359790 | Stroke | A | G | 0.015353 | 0.130873 | A | G | −0.002419838 |
| rs1367117 | Stroke | G | A | 0.015255 | 0.130873 | A | G | 0.003706995 |
| rs13723 | Stroke | G | A | 0.028068 | 0.130873 | G | A | 0.016961219 |
| rs1412444 | Stroke | T | C | 0.020314 | 0.130873 | T | C | 0.019498927 |
| rs1436953 | Stroke | T | C | 0.016606 | 0.130873 | T | C | −0.000256013 |
| rs1470579 | Stroke | C | A | 0.022475 | 0.130873 | C | A | 0.026910624 |
| rs1495741 | Stroke | G | A | 0.016262 | 0.130873 | A | G | −0.014478301 |
| rs1508798 | Stroke | T | C | 0.023978 | 0.130873 | C | T | −0.00981614 |
| rs151193009 | Stroke | C | T | 0.077817 | 0.130873 | T | C | −0.139164839 |
| rs1552224 | Stroke | A | C | 0.02055 | 0.130873 | C | A | −0.020646215 |
| rs1591805 | Stroke | G | A | 0.049599 | 0.130873 | A | G | −0.026499447 |
| rs16844401 | Stroke | A | G | 0.024858 | 0.130873 | A | G | 0.02223344 |
| rs16849225 | Stroke | T | C | 0.014802 | 0.130873 | T | C | −0.000663936 |
| rs16858082 | Stroke | T | C | 0.017489 | 0.130873 | T | C | 0.018989763 |
| rs16896398 | Stroke | T | A | 0.036089 | 0.130873 | T | A | 0.041632023 |
| rs16967013 | Stroke | G | C | 0.025141 | 0.130873 | G | C | 0.012335164 |
| rs16999793 | Stroke | G | C | 0.029446 | 0.130873 | C | G | −0.021757907 |
| rs17030613 | Stroke | C | A | 0.034764 | 0.130873 | C | A | 0.016000458 |
| rs17080091 | Stroke | C | T | 0.050749 | 0.130873 | T | C | −0.023063404 |
| rs17087335 | Stroke | T | G | 0.023089 | 0.130873 | T | G | 0.019931657 |
| rs17122278 | Stroke | A | G | 0.019699 | 0.130873 | G | A | −8.16572E−05 |
| rs17135399 | Stroke | G | A | 0.030379 | 0.130873 | G | A | 0.021239921 |
| rs17301514 | Stroke | G | A | 0.016816 | 0.130873 | A | G | 0.008706071 |
| rs173396 | Stroke | A | G | 0.035287 | 0.130873 | A | G | 0.017574516 |
| rs17358402 | Stroke | C | T | 0.065603 | 0.130873 | T | C | −0.008476171 |
| rs17477177 | Stroke | C | T | 0.048979 | 0.130873 | C | T | 0.030188635 |
| rs17514846 | Stroke | A | C | 0.019188 | 0.130873 | A | C | 0.041376923 |
| rs17581137 | Stroke | C | A | 0.013486 | 0.130873 | C | A | 0.003580073 |
| rs17612742 | Stroke | C | T | 0.029018 | 0.130873 | C | T | 0.02827309 |
| rs17680741 | Stroke | C | T | 0.047696 | 0.130873 | C | T | 0.017286182 |
| rs17791513 | Stroke | G | A | 0.018134 | 0.130873 | G | A | −0.000413747 |
| rs180327 | Stroke | T | C | 0.018375 | 0.130873 | C | T | −0.002885431 |
| rs181359 | Stroke | A | G | 0.017294 | 0.130873 | A | G | 0.005030319 |
| rs1861411 | Stroke | G | A | 0.008532 | 0.130873 | A | G | 0.019235747 |
| rs1868673 | Stroke | C | A | 0.012633 | 0.130873 | A | C | −0.010408758 |
| rs1870634 | Stroke | T | G | 0.013898 | 0.130873 | T | G | 0.004229877 |
| rs1887320 | Stroke | A | G | 0.050599 | 0.130873 | G | A | −0.025805227 |
| rs1892094 | Stroke | C | T | 0.027706 | 0.130873 | T | C | −0.019559719 |
| rs1902859 | Stroke | C | T | 0.045169 | 0.130873 | C | T | 0.025912838 |
| rs191835914 | Stroke | A | C | 0.068835 | 0.130873 | C | A | −0.065336943 |
| rs1976041 | Stroke | G | A | 0.01304 | 0.130873 | A | G | −0.017424821 |
| rs1982963 | Stroke | A | G | 0.024547 | 0.130873 | G | A | −0.010760979 |
| rs2000813 | Stroke | C | T | 0.018586 | 0.130873 | T | C | −0.005342938 |
| rs2028299 | Stroke | C | A | 0.016888 | 0.130873 | C | A | 0.01063865 |
| rs2057291 | Stroke | G | A | 0.016896 | 0.130873 | A | G | −0.022533222 |
| rs2068888 | Stroke | G | A | 0.039646 | 0.130873 | G | A | 0.025356426 |
| rs2074158 | Stroke | C | T | 0.024477 | 0.130873 | C | T | 0.017848147 |
| rs2075291 | Stroke | A | C | 0.016119 | 0.130873 | A | C | 0.051598332 |
| rs2075423 | Stroke | G | T | 0.027418 | 0.130873 | T | G | −0.010934476 |
| rs2107595 | Stroke | A | G | 0.032357 | 0.130873 | A | G | 0.036440157 |
| rs2128739 | Stroke | C | A | 0.011793 | 0.130873 | C | A | −0.009494306 |
| rs2145598 | Stroke | A | G | 0.00835 | 0.130873 | A | G | −0.001431914 |
| rs216172 | Stroke | C | G | 0.023012 | 0.130873 | C | G | 0.01141003 |
| rs2213732 | Stroke | G | A | 0.019353 | 0.130873 | G | A | 0.010510482 |
| rs2229383 | Stroke | T | G | 0.022101 | 0.130873 | G | T | −0.015958752 |
| rs2237896 | Stroke | A | G | 0.012236 | 0.130873 | A | G | −0.019584506 |
| rs2240736 | Stroke | T | C | 0.038422 | 0.130873 | T | C | 0.030384134 |
| rs2245019 | Stroke | A | C | 0.023781 | 0.130873 | C | A | −0.008681144 |
| rs2261181 | Stroke | C | T | 0.01833 | 0.130873 | T | C | 5.90253E−05 |
| rs2295786 | Stroke | A | T | 0.071995 | 0.130873 | A | T | 0.028778918 |
| rs2334499 | Stroke | T | C | 0.030007 | 0.130873 | C | T | −0.016185775 |
| rs243019 | Stroke | T | C | 0.019535 | 0.130873 | T | C | 0.001389449 |
| rs246600 | Stroke | C | T | 0.040167 | 0.130873 | T | C | −0.019302412 |
| rs247616 | Stroke | C | T | 0.017696 | 0.130873 | T | C | −0.002767757 |
| rs2487928 | Stroke | A | G | 0.016775 | 0.130873 | A | G | 0.008166753 |
| rs2535633 | Stroke | G | C | 0.012232 | 0.130873 | G | C | 0.018491435 |
| rs2575876 | Stroke | G | A | 0.011366 | 0.130873 | A | G | −0.01271276 |
| rs261967 | Stroke | C | A | 0.010172 | 0.130873 | C | A | 0.013098379 |
| rs273909 | Stroke | G | A | 0.079753 | 0.130873 | G | A | 0.025161792 |
| rs2758607 | Stroke | G | A | 0.03978 | 0.130873 | A | G | −0.023813117 |
| rs2782980 | Stroke | T | C | 0.018405 | 0.130873 | T | C | 0.006009048 |
| rs2796441 | Stroke | A | G | 0.010432 | 0.130873 | G | A | 0.003598868 |
| rs2815752 | Stroke | G | A | 0.017329 | 0.130873 | G | A | 0.0130348 |
| rs2820315 | Stroke | C | T | 0.024314 | 0.130873 | T | C | −0.010369523 |
| rs2861568 | Stroke | T | A | 0.018315 | 0.130873 | T | A | 0.012588058 |
| rs2925979 | Stroke | T | C | 0.016411 | 0.130873 | T | C | 0.014501138 |
| rs2972146 | Stroke | G | T | 0.016939 | 0.130873 | G | T | −0.005336023 |
| rs29941 | Stroke | G | A | 0.014661 | 0.130873 | G | A | 0.01427765 |
| rs326214 | Stroke | A | G | 0.011612 | 0.130873 | A | G | 0.01918858 |
| rs340874 | Stroke | C | T | 0.01991 | 0.130873 | C | T | 0.01113197 |
| rs351855 | Stroke | A | G | 0.011838 | 0.130873 | A | G | −0.023236764 |
| rs35337492 | Stroke | A | G | 0.045528 | 0.130873 | A | G | 0.021699395 |
| rs35444 | Stroke | A | G | 0.048251 | 0.130873 | G | A | −0.059405505 |
| rs36096196 | Stroke | T | C | 0.014778 | 0.130873 | T | C | 0.019218893 |
| rs368123 | Stroke | G | A | 0.023167 | 0.130873 | G | A | 0.009484191 |
| rs376563 | Stroke | T | C | 0.025842 | 0.130873 | T | C | 0.007620112 |
| rs3775058 | Stroke | A | T | 0.021506 | 0.130873 | T | A | −0.016346198 |
| rs3785100 | Stroke | T | C | 0.024126 | 0.130873 | C | T | 0.000791873 |
| rs3791679 | Stroke | G | A | 0.014504 | 0.130873 | A | G | 0.044064697 |
| rs3861086 | Stroke | C | T | 0.013681 | 0.130873 | T | C | −0.010211566 |
| rs3887137 | Stroke | C | T | 0.016126 | 0.130873 | T | C | 0.001835572 |
| rs3903239 | Stroke | G | A | 0.024173 | 0.130873 | A | G | −0.013596237 |
| rs3936511 | Stroke | G | A | 0.015486 | 0.130873 | G | A | 0.009894547 |
| rs4275659 | Stroke | T | C | 0.0158 | 0.130873 | T | C | 0.004215837 |
| rs4400058 | Stroke | G | A | 0.0101 | 0.130873 | A | G | −0.006059144 |
| rs4409766 | Stroke | C | T | 0.028526 | 0.130873 | C | T | −0.020930043 |
| rs4458523 | Stroke | G | T | 0.041816 | 0.130873 | T | G | −0.031606827 |
| rs4468572 | Stroke | T | C | 0.020931 | 0.130873 | T | C | −0.008664437 |
| rs4593108 | Stroke | C | G | 0.013345 | 0.130873 | G | C | −0.015338107 |
| rs46522 | Stroke | T | C | 0.019506 | 0.130873 | C | T | −0.019302164 |
| rs4719841 | Stroke | G | A | 0.008792 | 0.130873 | A | G | −0.001669583 |
| rs4722766 | Stroke | C | G | 0.01922 | 0.130873 | C | G | −0.000162089 |
| rs4724806 | Stroke | C | G | 0.026351 | 0.130873 | G | C | −0.015422077 |
| rs4731420 | Stroke | C | G | 0.026014 | 0.130873 | C | G | 0.016028248 |
| rs4752700 | Stroke | G | A | 0.009114 | 0.130873 | G | A | 0.011750063 |
| rs4766228 | Stroke | A | G | 0.029202 | 0.130873 | A | G | 0.019459337 |
| rs4788102 | Stroke | A | G | 0.018717 | 0.130873 | A | G | 0.022539971 |
| rs4812829 | Stroke | G | A | 0.008682 | 0.130873 | A | G | 0.003826629 |
| rs4821382 | Stroke | G | C | 0.015065 | 0.130873 | G | C | 0.012565531 |
| rs4836831 | Stroke | C | T | 0.012074 | 0.130873 | C | T | 0.023306095 |
| rs4846049 | Stroke | G | T | 0.03021 | 0.130873 | T | G | −0.01659793 |
| rs4883263 | Stroke | T | C | 0.025599 | 0.130873 | T | C | 0.008395136 |
| rs4911495 | Stroke | A | C | 0.011258 | 0.130873 | C | A | −0.005940987 |
| rs4918072 | Stroke | A | G | 0.049832 | 0.130873 | A | G | 0.019418054 |
| rs4932370 | Stroke | A | G | 0.027973 | 0.130873 | A | G | 0.011905353 |
| rs556621 | Stroke | T | G | 0.024962 | 0.130873 | T | G | 0.01346888 |
| rs56062135 | Stroke | C | T | 0.047243 | 0.130873 | T | C | −0.083012494 |
| rs574367 | Stroke | T | G | 0.026224 | 0.130873 | T | G | 0.04417131 |
| rs579459 | Stroke | T | C | 0.017538 | 0.130873 | C | T | 0.002391067 |
| rs582384 | Stroke | A | C | 0.012376 | 0.130873 | A | C | 0.008267904 |
| rs5996074 | Stroke | G | A | 0.024973 | 0.130873 | G | A | 0.006793319 |
| rs6093446 | Stroke | G | A | 0.016236 | 0.130873 | A | G | −0.001469287 |
| rs61776719 | Stroke | A | C | 0.009033 | 0.130873 | A | C | 0.01110414 |
| rs633185 | Stroke | G | C | 0.008645 | 0.130873 | C | G | 0.022775046 |
| rs6490029 | Stroke | A | G | 0.065799 | 0.130873 | G | A | −0.060472816 |
| rs6545814 | Stroke | A | G | 0.015714 | 0.130873 | G | A | −0.002951895 |
| rs663129 | Stroke | A | G | 0.021433 | 0.130873 | A | G | 0.03350231 |
| rs6666258 | Stroke | C | G | 0.040122 | 0.130873 | C | G | 0.007059752 |
| rs667920 | Stroke | G | T | 0.022184 | 0.130873 | G | T | 0.001829257 |
| rs6700559 | Stroke | C | T | 0.015616 | 0.130873 | C | T | 0.013815903 |
| rs671 | Stroke | G | A | 0.118327 | 0.130873 | A | G | −0.059610664 |
| rs67156297 | Stroke | A | G | 0.014525 | 0.130873 | A | G | 0.017918676 |
| rs67180937 | Stroke | G | T | 0.012499 | 0.130873 | T | G | −0.004490533 |
| rs6725887 | Stroke | T | C | 0.050075 | 0.130873 | C | T | −0.008832831 |
| rs67839313 | Stroke | T | C | 0.029265 | 0.130873 | C | T | −0.004645941 |
| rs6795735 | Stroke | C | T | 0.014651 | 0.130873 | C | T | −0.00267444 |
| rs6813195 | Stroke | C | T | 0.01888 | 0.130873 | T | C | −0.015190778 |
| rs6817105 | Stroke | C | T | 0.034557 | 0.130873 | T | C | −0.012825256 |
| rs6825454 | Stroke | C | T | 0.048175 | 0.130873 | C | T | 0.022614059 |
| rs6825911 | Stroke | C | T | 0.032991 | 0.130873 | C | T | 0.026500756 |
| rs6829822 | Stroke | T | G | 0.026941 | 0.130873 | T | G | 0.02638728 |
| rs6831256 | Stroke | G | A | 0.01504 | 0.130873 | G | A | 0.014547578 |
| rs6838973 | Stroke | C | T | 0.040631 | 0.130873 | C | T | 0.023223346 |
| rs6878122 | Stroke | A | G | 0.134586 | 0.130873 | G | A | −0.055227705 |
| rs6882076 | Stroke | T | C | 0.014248 | 0.130873 | T | C | −0.004725523 |
| rs6905288 | Stroke | A | G | 0.01558 | 0.130873 | G | A | −0.017852116 |
| rs6909752 | Stroke | A | G | 0.011675 | 0.130873 | A | G | 0.02230427 |
| rs6960043 | Stroke | C | T | 0.022526 | 0.130873 | C | T | 0.020004241 |
| rs699 | Stroke | G | A | 0.024286 | 0.130873 | A | G | −0.011680682 |
| rs6997340 | Stroke | T | C | 0.017152 | 0.130873 | C | T | −0.006813863 |
| rs702485 | Stroke | A | G | 0.050584 | 0.130873 | A | G | 0.033319746 |
| rs702634 | Stroke | A | G | 0.010333 | 0.130873 | G | A | −0.014191055 |
| rs7136259 | Stroke | C | T | 0.055595 | 0.130873 | T | C | −0.029059864 |
| rs7164883 | Stroke | A | G | 0.048442 | 0.130873 | G | A | −0.019478575 |
| rs7178572 | Stroke | A | G | 0.00927 | 0.130873 | G | A | 0.010484018 |
| rs7193343 | Stroke | T | C | 0.03008 | 0.130873 | C | T | −0.015964015 |
| rs7199941 | Stroke | G | A | 0.013461 | 0.130873 | A | G | −0.001088974 |
| rs7202877 | Stroke | T | G | 0.043416 | 0.130873 | G | T | −0.024280978 |
| rs7206541 | Stroke | T | A | 0.038382 | 0.130873 | A | T | −0.03118216 |
| rs7258189 | Stroke | T | C | 0.036369 | 0.130873 | C | T | −0.012802341 |
| rs7258445 | Stroke | G | A | 0.024739 | 0.130873 | A | G | −0.013392482 |
| rs7258950 | Stroke | A | G | 0.011268 | 0.130873 | A | G | −0.005481263 |
| rs72689147 | Stroke | G | T | 0.016558 | 0.130873 | T | G | −0.014591515 |
| rs73015714 | Stroke | G | C | 0.089997 | 0.130873 | G | C | 0.043674893 |
| rs7304841 | Stroke | A | C | 0.059234 | 0.130873 | C | A | −0.026809054 |
| rs7306455 | Stroke | G | A | 0.011301 | 0.130873 | A | G | −0.004809562 |
| rs73069940 | Stroke | G | C | 0.022048 | 0.130873 | G | C | −0.004463573 |
| rs736699 | Stroke | A | G | 0.073129 | 0.130873 | G | A | −0.037618244 |
| rs737337 | Stroke | T | C | 0.013933 | 0.130873 | C | T | −0.01369532 |
| rs7403531 | Stroke | T | C | 0.0204 | 0.130873 | T | C | 0.016541069 |
| rs740406 | Stroke | G | A | 0.024747 | 0.130873 | G | A | 0.024212946 |
| rs7499892 | Stroke | T | C | 0.03885 | 0.130873 | T | C | 0.011652376 |
| rs7500448 | Stroke | A | G | 0.019974 | 0.130873 | G | A | −0.020954494 |
| rs7503807 | Stroke | A | C | 0.019568 | 0.130873 | C | A | −0.017737968 |
| rs7568458 | Stroke | T | A | 0.035295 | 0.130873 | A | T | −0.006929591 |
| rs7610618 | Stroke | C | T | 0.068132 | 0.130873 | T | C | −0.024025783 |
| rs7616006 | Stroke | A | G | 0.009588 | 0.130873 | G | A | −0.003311607 |
| rs7696431 | Stroke | G | T | 0.013095 | 0.130873 | T | G | −0.002030321 |
| rs7770628 | Stroke | C | T | 0.01507 | 0.130873 | C | T | 0.029882698 |
| rs780094 | Stroke | T | C | 0.011455 | 0.130873 | C | T | −0.010074842 |
| rs7810507 | Stroke | A | G | 0.015223 | 0.130873 | A | G | 0.012560154 |
| rs7859727 | Stroke | T | C | 0.027481 | 0.130873 | C | T | −0.011695784 |
| rs7917772 | Stroke | G | A | 0.011413 | 0.130873 | A | G | −0.001357453 |
| rs79223353 | Stroke | G | A | 0.020516 | 0.130873 | A | G | −0.014227275 |
| rs7947761 | Stroke | G | A | 0.01936 | 0.130873 | G | A | 0.028555911 |
| rs7955901 | Stroke | C | T | 0.014767 | 0.130873 | T | C | −0.008203819 |
| rs7965082 | Stroke | C | T | 0.016959 | 0.130873 | T | C | −0.013197172 |
| rs7980458 | Stroke | G | T | 0.016676 | 0.130873 | G | T | 0.014840736 |
| rs8042271 | Stroke | A | G | 0.014211 | 0.130873 | A | G | −0.004002873 |
| rs8108269 | Stroke | T | G | 0.013902 | 0.130873 | T | G | −0.005058046 |
| rs838880 | Stroke | C | T | 0.016019 | 0.130873 | T | C | −0.004594303 |
| rs840616 | Stroke | C | T | 0.018957 | 0.130873 | T | C | −0.017683048 |
| rs871606 | Stroke | T | C | 0.020852 | 0.130873 | C | T | −0.032441283 |
| rs880315 | Stroke | C | T | 0.045163 | 0.130873 | T | C | −0.052414405 |
| rs884366 | Stroke | A | G | 0.015561 | 0.130873 | A | G | 0.008102981 |
| rs885150 | Stroke | C | T | 0.019994 | 0.130873 | C | T | 0.012125497 |
| rs888789 | Stroke | G | A | 0.012833 | 0.130873 | A | G | −0.001929693 |
| rs9266359 | Stroke | C | T | 0.034031 | 0.130873 | T | C | −0.028754368 |
| rs9268402 | Stroke | G | A | 0.010467 | 0.130873 | A | G | −0.006782196 |
| rs9299 | Stroke | T | C | 0.017512 | 0.130873 | C | T | −0.015005794 |
| rs9319428 | Stroke | G | A | 0.010582 | 0.130873 | A | G | 0.000838789 |
| rs9376090 | Stroke | C | T | 0.013568 | 0.130873 | C | T | 0.00571639 |
| rs9473924 | Stroke | T | G | 0.010882 | 0.130873 | T | G | 0.01349432 |
| rs9505118 | Stroke | G | A | 0.008154 | 0.130873 | G | A | 0.007041858 |
| rs9568867 | Stroke | A | G | 0.01304 | 0.130873 | A | G | 0.013855392 |
| rs964184 | Stroke | C | G | 0.016164 | 0.130873 | G | C | −0.000924239 |
| rs9687065 | Stroke | G | A | 0.012681 | 0.130873 | G | A | −0.014943928 |
| rs975722 | Stroke | G | A | 0.022427 | 0.130873 | G | A | 0.017303635 |
| rs9810888 | Stroke | G | T | 0.028825 | 0.130873 | G | T | 0.031266759 |
| rs9815354 | Stroke | G | A | 0.035293 | 0.130873 | A | G | −0.019701091 |
| rs9828933 | Stroke | C | T | 0.013198 | 0.130873 | C | T | −0.002640433 |
| rs984222 | Stroke | C | G | 0.012832 | 0.130873 | C | G | −0.020995148 |
| rs9892152 | Stroke | C | T | 0.015381 | 0.130873 | T | C | −0.013102488 |
| rs9970807 | Stroke | T | C | 0.048092 | 0.130873 | T | C | 0.00491025 |
| rs11847697 | CAD | C | T | 0.5391 | 0.090296 | T | C | −0.054854254 |
| rs72654473 | CAD | C | A | 0.1981 | 0.090296 | A | C | −0.044900213 |
| rs3809128 | CAD | T | C | 0.0556 | 0.090296 | T | C | −0.044258299 |
| rs11066280 | CAD | A | T | 0.024 | 0.090296 | A | T | −0.043499545 |
| rs12740374 | CAD | G | T | 0.166 | 0.090296 | T | G | −0.035270252 |
| rs17517928 | CAD | C | T | 0.4302 | 0.090296 | T | C | −0.034680524 |
| rs8030379 | CAD | A | G | 0.0334 | 0.090296 | G | A | −0.034258012 |
| rs10203174 | CAD | C | T | 0.3286 | 0.090296 | T | C | −0.029405592 |
| rs10513801 | CAD | T | G | 0.1378 | 0.090296 | G | T | −0.028358809 |
| rs9357121 | CAD | T | G | 0.2308 | 0.090296 | G | T | −0.026951882 |
| rs12463617 | CAD | C | A | 0.0497 | 0.090296 | A | C | −0.024810856 |
| rs11030104 | CAD | A | G | 0.0518 | 0.090296 | G | A | −0.024808821 |
| rs1575972 | CAD | T | A | 0.0966 | 0.090296 | A | T | −0.023269228 |
| rs13306194 | CAD | G | A | 0.096 | 0.090296 | A | G | −0.022882798 |
| rs12999907 | CAD | A | G | 0.0416 | 0.090296 | G | A | −0.022380593 |
| rs17381664 | CAD | T | C | 0.1911 | 0.090296 | C | T | −0.021861111 |
| rs10096633 | CAD | C | T | 0.1174 | 0.090296 | T | C | −0.021775395 |
| rs3993105 | CAD | T | C | 0.0288 | 0.090296 | C | T | −0.021611657 |
| rs17080102 | CAD | G | C | 0.1439 | 0.090296 | C | G | −0.020058605 |
| rs17249754 | CAD | A | G | 0.1082 | 0.090296 | A | G | −0.01837771 |
| rs1378942 | CAD | C | A | 0.0399 | 0.090296 | A | C | −0.018302246 |
| rs2383208 | CAD | G | A | 0.0293 | 0.090296 | G | A | −0.017615663 |
| rs12597579 | CAD | C | T | 0.0529 | 0.090296 | T | C | −0.017560397 |
| rs12524865 | CAD | C | A | 0.1436 | 0.090296 | A | C | −0.017231543 |
| rs9349379 | CAD | G | A | 0.1856 | 0.090296 | A | G | −0.016792767 |
| rs13266634 | CAD | C | T | 0.0245 | 0.090296 | T | C | −0.015438749 |
| rs10401969 | CAD | T | C | 0.0856 | 0.090296 | C | T | −0.015394143 |
| rs897057 | CAD | C | T | 0.0613 | 0.090296 | T | C | −0.013906254 |
| rs7901016 | CAD | T | C | 0.0853 | 0.090296 | C | T | −0.013637478 |
| rs4377290 | CAD | T | C | 0.0661 | 0.090296 | C | T | −0.013595416 |
| rs10842992 | CAD | T | C | 0.0574 | 0.090296 | C | T | −0.01313334 |
| rs11556924 | CAD | C | T | 0.0982 | 0.090296 | T | C | −0.012459898 |
| rs2021783 | CAD | C | T | 0.1009 | 0.090296 | T | C | −0.01145951 |
| rs4923678 | CAD | A | G | 0.0881 | 0.090296 | G | A | −0.011429339 |
| rs17465637 | CAD | C | A | 0.0861 | 0.090296 | A | C | −0.011236064 |
| rs11205760 | CAD | T | C | 0.0152 | 0.090296 | C | T | −0.010275887 |
| rs2302593 | CAD | C | G | 0.0298 | 0.090296 | G | C | −0.010062 |
| rs3846663 | CAD | T | C | 0.0244 | 0.090296 | C | T | −0.009905924 |
| rs1800234 | CAD | T | C | 0.0362 | 0.090296 | C | T | −0.009847287 |
| rs6065311 | CAD | C | T | 0.0245 | 0.090296 | T | C | −0.009578482 |
| rs1496653 | CAD | G | A | 0.0263 | 0.090296 | G | A | −0.009311541 |
| rs181360 | CAD | T | G | 0.0852 | 0.090296 | G | T | −0.008669231 |
| rs11125936 | CAD | T | C | 0.0426 | 0.090296 | C | T | −0.008668353 |
| rs1801282 | CAD | G | C | 0.0617 | 0.090296 | G | C | −0.008172862 |
| rs11067763 | CAD | A | G | 0.0675 | 0.090296 | G | A | −0.007334401 |
| rs11838267 | CAD | T | C | 0.1211 | 0.090296 | C | T | −0.007293479 |
| rs4713766 | CAD | C | A | 0.078 | 0.090296 | A | C | −0.007025249 |
| rs660599 | CAD | G | A | 0.0506 | 0.090296 | A | G | −0.006967027 |
| rs12970066 | CAD | C | G | 0.0397 | 0.090296 | G | C | −0.006678446 |
| rs56336142 | CAD | T | C | 0.0418 | 0.090296 | C | T | −0.006196103 |
| rs7525649 | CAD | T | C | 0.0575 | 0.090296 | C | T | −0.00616764 |
| rs4266144 | CAD | G | C | 0.0641 | 0.090296 | C | G | −0.00615486 |
| rs7617773 | CAD | T | C | 0.047 | 0.090296 | C | T | −0.005907439 |
| rs748431 | CAD | G | T | 0.0255 | 0.090296 | T | G | −0.005765377 |
| rs2066714 | CAD | C | T | 0.0524 | 0.090296 | T | C | −0.00575392 |
| rs2144300 | CAD | C | T | 0.0362 | 0.090296 | T | C | −0.005568953 |
| rs11136341 | CAD | A | G | 0.0368 | 0.090296 | G | A | −0.005551974 |
| rs13277801 | CAD | T | C | 0.0671 | 0.090296 | C | T | −0.005375349 |
| rs12679556 | CAD | G | T | 0.031 | 0.090296 | T | G | −0.004964713 |
| rs1514175 | CAD | A | G | 0.0359 | 0.090296 | G | A | −0.004948626 |
| rs867186 | CAD | A | G | 0.0739 | 0.090296 | G | A | −0.004895106 |
| rs9552911 | CAD | G | A | 0.0332 | 0.090296 | A | G | −0.004507845 |
| rs174547 | CAD | T | C | 0.0153 | 0.090296 | C | T | −0.004304546 |
| rs2642442 | CAD | T | C | 0.023 | 0.090296 | C | T | −0.004286239 |
| rs2075260 | CAD | A | G | 0.016 | 0.090296 | G | A | −0.00398427 |
| rs8090011 | CAD | C | G | 0.0636 | 0.090296 | C | G | −0.003761664 |
| rs995000 | CAD | C | T | 0.0367 | 0.090296 | T | C | −0.003734281 |
| rs2237892 | CAD | C | T | 0.0437 | 0.090296 | T | C | −0.002759778 |
| rs6494488 | CAD | A | G | 0.0452 | 0.090296 | G | A | −0.002733586 |
| rs4471613 | CAD | A | G | 0.0347 | 0.090296 | A | G | −0.002688648 |
| rs3130501 | CAD | A | G | 0.0328 | 0.090296 | A | G | −0.002329046 |
| rs16990971 | CAD | A | G | 0.0488 | 0.090296 | G | A | −0.0022166 |
| rs11142387 | CAD | A | C | 0.0198 | 0.090296 | C | A | −0.002103931 |
| rs12535846 | CAD | G | A | 0.0182 | 0.090296 | A | G | −0.001851878 |
| rs4613862 | CAD | A | C | 0.0552 | 0.090296 | C | A | −0.00183526 |
| rs17150703 | CAD | G | A | 0.0319 | 0.090296 | A | G | −0.00175232 |
| rs12242953 | CAD | G | A | 0.048 | 0.090296 | A | G | −0.001702692 |
| rs2415317 | CAD | G | A | 0.0181 | 0.090296 | A | G | −0.00063107 |
| rs1027087 | CAD | T | A | 0.019 | 0.090296 | T | A | −0.000407661 |
| rs3213545 | CAD | A | G | 0.0224 | 0.090296 | A | G | −0.000326043 |
| rs4757391 | CAD | C | T | 0.0336 | 0.090296 | C | T | −0.000163507 |
| rs9309245 | CAD | C | G | 0.0401 | 0.090296 | G | C | 0.000135511 |
| rs499974 | CAD | A | C | 0.0139 | 0.090296 | A | C | 0.000259526 |
| rs12453914 | CAD | C | A | 0.0122 | 0.090296 | A | C | 0.000304355 |
| rs13041126 | CAD | C | T | 0.0307 | 0.090296 | C | T | 0.000322866 |
| rs11830157 | CAD | T | G | 0.0181 | 0.090296 | G | T | 0.000413219 |
| rs1211166 | CAD | G | A | 0.0263 | 0.090296 | G | A | 0.000426445 |
| rs439401 | CAD | T | C | 0.014 | 0.090296 | C | T | 0.000924731 |
| rs16933812 | CAD | G | T | 0.0361 | 0.090296 | G | T | 0.001818536 |
| rs9593 | CAD | T | A | 0.0184 | 0.090296 | T | A | 0.001872228 |
| rs4917014 | CAD | T | G | 0.0248 | 0.090296 | G | T | 0.001912177 |
| rs35332062 | CAD | A | G | 0.0351 | 0.090296 | A | G | 0.002027526 |
| rs11634397 | CAD | G | A | 0.0362 | 0.090296 | G | A | 0.002140488 |
| rs7989336 | CAD | A | G | 0.0136 | 0.090296 | A | G | 0.002368031 |
| rs9390698 | CAD | A | G | 0.0345 | 0.090296 | A | G | 0.002507997 |
| rs11057830 | CAD | A | G | 0.0346 | 0.090296 | A | G | 0.002601537 |
| rs4845625 | CAD | T | C | 0.0209 | 0.090296 | T | C | 0.002666252 |
| rs10886471 | CAD | T | C | 0.0177 | 0.090296 | T | C | 0.00274217 |
| rs17678683 | CAD | G | T | 0.0575 | 0.090296 | G | T | 0.002869027 |
| rs1555543 | CAD | A | C | 0.0275 | 0.090296 | A | C | 0.004136923 |
| rs590121 | CAD | T | G | 0.0637 | 0.090296 | T | G | 0.004236743 |
| rs10830963 | CAD | G | C | 0.0167 | 0.090296 | G | C | 0.004251081 |
| rs2571445 | CAD | A | G | 0.045 | 0.090296 | A | G | 0.004345257 |
| rs4148008 | CAD | G | C | 0.0243 | 0.090296 | G | C | 0.004385903 |
| rs10820405 | CAD | A | G | 0.0184 | 0.090296 | A | G | 0.004424645 |
| rs11077501 | CAD | C | T | 0.0242 | 0.090296 | C | T | 0.004486902 |
| rs2043085 | CAD | T | C | 0.0442 | 0.090296 | T | C | 0.004497418 |
| rs9534262 | CAD | T | C | 0.0244 | 0.090296 | T | C | 0.005057555 |
| rs1689800 | CAD | G | A | 0.0259 | 0.090296 | G | A | 0.005133146 |
| rs9367716 | CAD | G | T | 0.013 | 0.090296 | G | T | 0.005277741 |
| rs2296172 | CAD | A | G | 0.0126 | 0.090296 | G | A | 0.005624046 |
| rs17695224 | CAD | A | G | 0.0252 | 0.090296 | A | G | 0.005722462 |
| rs944172 | CAD | C | T | 0.0473 | 0.090296 | C | T | 0.005882857 |
| rs55783344 | CAD | T | C | 0.0184 | 0.090296 | T | C | 0.006100025 |
| rs2820443 | CAD | C | T | 0.0787 | 0.090296 | C | T | 0.006163185 |
| rs459193 | CAD | G | A | 0.037 | 0.090296 | A | G | 0.006188951 |
| rs10455782 | CAD | T | C | 0.0459 | 0.090296 | T | C | 0.006509473 |
| rs7116641 | CAD | G | T | 0.0146 | 0.090296 | G | T | 0.00653143 |
| rs1467605 | CAD | A | C | 0.0927 | 0.090296 | A | C | 0.006693304 |
| rs1129555 | CAD | A | G | 0.0734 | 0.090296 | A | G | 0.006793106 |
| rs2328223 | CAD | C | A | 0.0353 | 0.090296 | C | A | 0.007169671 |
| rs1029420 | CAD | C | T | 0.0961 | 0.090296 | C | T | 0.008397498 |
| rs2106261 | CAD | T | C | 0.0522 | 0.090296 | T | C | 0.00841714 |
| rs7225581 | CAD | A | T | 0.0586 | 0.090296 | A | T | 0.008470473 |
| rs2531995 | CAD | T | C | 0.014 | 0.090296 | T | C | 0.008612813 |
| rs391300 | CAD | T | C | 0.0646 | 0.090296 | T | C | 0.008665579 |
| rs17609940 | CAD | C | G | 0.1255 | 0.090296 | C | G | 0.009008416 |
| rs9470794 | CAD | C | T | 0.0188 | 0.090296 | C | T | 0.009198202 |
| rs1169288 | CAD | C | A | 0.0306 | 0.090296 | C | A | 0.009641797 |
| rs16986953 | CAD | A | G | 0.0969 | 0.090296 | A | G | 0.009859724 |
| rs1535500 | CAD | G | T | 0.0132 | 0.090296 | T | G | 0.009900765 |
| rs6818397 | CAD | T | G | 0.0284 | 0.090296 | T | G | 0.009902368 |
| rs1317507 | CAD | A | C | 0.0547 | 0.090296 | A | C | 0.00990365 |
| rs896854 | CAD | T | C | 0.0607 | 0.090296 | T | C | 0.010003728 |
| rs5215 | CAD | C | T | 0.0377 | 0.090296 | C | T | 0.010020025 |
| rs2819348 | CAD | C | T | 0.1056 | 0.090296 | C | T | 0.010744962 |
| rs4420638 | CAD | G | A | 0.094 | 0.090296 | G | A | 0.011063463 |
| rs1799945 | CAD | G | C | 0.0432 | 0.090296 | G | C | 0.011981088 |
| rs6807945 | CAD | C | T | 0.1069 | 0.090296 | C | T | 0.012288749 |
| rs7107784 | CAD | A | G | 0.0247 | 0.090296 | G | A | 0.012508937 |
| rs3129853 | CAD | A | G | 0.0435 | 0.090296 | A | G | 0.013553305 |
| rs2000999 | CAD | A | G | 0.058 | 0.090296 | A | G | 0.014119748 |
| rs17843768 | CAD | A | C | 0.1021 | 0.090296 | A | C | 0.014655356 |
| rs1077834 | CAD | C | T | 0.0569 | 0.090296 | C | T | 0.014755658 |
| rs3827066 | CAD | T | C | 0.1357 | 0.090296 | T | C | 0.01479976 |
| rs4735692 | CAD | A | G | 0.0394 | 0.090296 | A | G | 0.017379708 |
| rs7087591 | CAD | G | A | 0.0335 | 0.090296 | G | A | 0.018541527 |
| rs56289821 | CAD | A | G | 0.1854 | 0.090296 | A | G | 0.018864735 |
| rs93138 | CAD | G | T | 0.065 | 0.090296 | G | T | 0.024665853 |
| rs13078807 | CAD | G | A | 0.1807 | 0.090296 | G | A | 0.024834421 |
| rs820430 | CAD | A | G | 0.0143 | 0.090296 | A | G | 0.025059737 |
| rs6808574 | CAD | T | C | 0.2478 | 0.090296 | T | C | 0.025214031 |
| rs651821 | CAD | C | T | 0.0899 | 0.090296 | C | T | 0.025338367 |
| rs130071 | CAD | A | G | 0.1063 | 0.090296 | A | G | 0.029473862 |
| rs12214416 | CAD | A | T | 0.1921 | 0.090296 | A | T | 0.029652614 |
| rs1558902 | CAD | A | T | 0.0804 | 0.090296 | A | T | 0.033351248 |
| rs78169666 | CAD | C | A | 1.8119 | 0.090296 | C | A | 0.170064403 |
| rs200990725 | CAD | T | C | 1.4136 | 0.090296 | T | C | 0.219305038 |
| rs12204590 | CAD | A | T | 3.5709 | 0.090296 | A | T | 0.363344567 |
| rs1275988 | SBP | C | T | 0.04269 | 0.070386 | T | C | −0.036252792 |
| rs7405452 | SBP | C | T | 0.0226 | 0.070386 | T | C | −0.024518448 |
| rs751984 | SBP | T | C | 0.01703 | 0.070386 | C | T | −0.015944358 |
| rs7701094 | SBP | C | G | 0.01826 | 0.070386 | C | G | 0.018406138 |
| rs2303790 | WC | A | G | 0.0632 | 0.067311 | G | A | −0.068928614 |
| rs9501744 | T2D | C | T | 0.1088 | 0.051256 | T | C | −0.011128426 |
| rs806215 | T2D | C | T | 0.0851 | 0.051256 | T | C | −0.010089649 |
| rs1260326 | T2D | C | T | 0.0679 | 0.051256 | C | T | −0.005623886 |
| rs2230808 | T2D | C | T | 0.013 | 0.051256 | T | C | −0.005378779 |
| rs2783963 | T2D | G | A | 0.0198 | 0.051256 | A | G | −0.005337994 |
| rs1052053 | T2D | A | G | 0.0344 | 0.051256 | G | A | −0.005163642 |
| rs42039 | T2D | C | T | 0.028 | 0.051256 | T | C | −0.005030576 |
| rs79548680 | T2D | C | G | 0.0547 | 0.051256 | G | C | −0.003823876 |
| rs10064156 | T2D | T | C | 0.0304 | 0.051256 | C | T | −0.003600503 |
| rs174546 | T2D | C | T | 0.0346 | 0.051256 | T | C | −0.003539003 |
| rs1832007 | T2D | G | A | 0.0181 | 0.051256 | G | A | −0.002967615 |
| rs7185272 | T2D | C | G | 0.0267 | 0.051256 | G | C | −0.002114669 |
| rs16927668 | T2D | T | C | 0.0111 | 0.051256 | C | T | −0.002095903 |
| rs58542926 | T2D | T | C | 0.0508 | 0.051256 | T | C | −0.002019295 |
| rs738409 | T2D | G | C | 0.0294 | 0.051256 | G | C | −0.001978904 |
| rs17843797 | T2D | T | G | 0.0192 | 0.051256 | G | T | −0.00196384 |
| rs4883201 | T2D | A | G | 0.0083 | 0.051256 | G | A | −0.001735897 |
| rs6038557 | T2D | A | G | 0.0196 | 0.051256 | G | A | −0.001234973 |
| rs3807989 | T2D | G | A | 0.0097 | 0.051256 | A | G | −0.001154249 |
| rs9591012 | T2D | A | G | 0.0148 | 0.051256 | A | G | −0.000126082 |
| rs60154123 | T2D | C | T | 0.0077 | 0.051256 | T | C | −0.000111693 |
| rs7528419 | T2D | G | A | 0.0203 | 0.051256 | G | A | 7.46602E−05 |
| rs9818870 | T2D | T | C | 0.0253 | 0.051256 | T | C | 0.000304788 |
| rs13143871 | T2D | C | T | 0.0229 | 0.051256 | C | T | 0.000505741 |
| rs1448818 | T2D | A | C | 0.0144 | 0.051256 | C | A | 0.000592428 |
| rs76954792 | T2D | T | C | 0.0255 | 0.051256 | T | C | 0.001032191 |
| rs9512699 | T2D | G | A | 0.033 | 0.051256 | G | A | 0.001269746 |
| rs10010670 | T2D | G | A | 0.0137 | 0.051256 | G | A | 0.001401282 |
| rs1800588 | T2D | C | T | 0.0205 | 0.051256 | T | C | 0.001532085 |
| rs2156552 | T2D | A | T | 0.0154 | 0.051256 | A | T | 0.001575163 |
| rs10923931 | T2D | T | G | 0.0352 | 0.051256 | T | G | 0.001802983 |
| rs2123536 | T2D | T | C | 0.03 | 0.051256 | T | C | 0.00180901 |
| rs2297991 | T2D | C | T | 0.0255 | 0.051256 | T | C | 0.00221143 |
| rs7678555 | T2D | C | A | 0.0172 | 0.051256 | C | A | 0.002326894 |
| rs4142995 | T2D | T | G | 0.0076 | 0.051256 | G | T | 0.002415799 |
| rs11660468 | T2D | C | T | 0.0106 | 0.051256 | T | C | 0.002894892 |
| rs7897379 | T2D | C | T | 0.0116 | 0.051256 | C | T | 0.003239983 |
| rs7208487 | T2D | G | T | 0.0404 | 0.051256 | G | T | 0.003240064 |
| rs634501 | T2D | A | G | 0.0388 | 0.051256 | A | G | 0.004097175 |
| rs7213603 | T2D | C | T | 0.0155 | 0.051256 | C | T | 0.004180513 |
| rs4302748 | T2D | A | G | 0.021 | 0.051256 | A | G | 0.004334334 |
| rs2258287 | T2D | C | A | 0.0457 | 0.051256 | C | A | 0.004674348 |
| rs6871667 | T2D | G | A | 0.0453 | 0.051256 | G | A | 0.004722276 |
| rs2081687 | T2D | C | T | 0.0116 | 0.051256 | T | C | 0.005005329 |
| rs6984210 | T2D | G | C | 0.049 | 0.051256 | G | C | 0.005011883 |
| rs17608766 | T2D | C | T | 0.0648 | 0.051256 | C | T | 0.00662796 |
| rs1532085 | T2D | G | A | 0.012 | 0.051256 | A | G | 0.007350074 |
| rs3810291 | T2D | A | G | 0.0218 | 0.051256 | A | G | 0.007667305 |
| rs80234489 | T2D | C | A | 0.0983 | 0.051256 | C | A | 0.007731913 |
| rs4776970 | T2D | A | T | 0.0375 | 0.051256 | A | T | 0.008887683 |
| rs2954029 | T2D | T | A | 0.0339 | 0.051256 | A | T | 0.009622163 |
| rs11651052 | T2D | A | G | 0.1161 | 0.051256 | A | G | 0.010599743 |
| rs769449 | T2D | G | A | 0.0364 | 0.051256 | A | G | 0.015579821 |
| rs7903146 | T2D | T | C | 0.281 | 0.051256 | T | C | 0.031385735 |
| rs3918226 | T2D | T | C | 2 | 0.051256 | T | C | 0.204566655 |
| rs10889353 | TC | A | C | 0.05622 | 0.035156 | C | A | −0.010122953 |
| rs1883025 | TC | C | T | 0.068547 | 0.035156 | T | C | −0.009566498 |
| rs3184504 | TC | C | T | 0.06665 | 0.035156 | T | C | −0.00742876 |
| rs9663362 | TC | C | G | 0.013 | 0.035156 | G | C | −0.002531662 |
| rs1805081 | TC | T | C | 0.013994 | 0.035156 | C | T | −0.002527985 |
| rs2625967 | TC | A | G | 0.0083 | 0.035156 | G | A | −0.002394552 |
| rs7306523 | TC | A | G | 0.01847 | 0.035156 | G | A | −0.002058652 |
| rs4939883 | TC | C | T | 0.04073 | 0.035156 | T | C | −0.001743175 |
| rs2972143 | TC | A | G | 0.0291 | 0.035156 | A | G | −0.001732713 |
| rs7633770 | TC | G | A | 0.0147 | 0.035156 | A | G | −0.001638451 |
| rs34008534 | TC | G | A | 0.004469 | 0.035156 | G | A | −0.001313211 |
| rs13115759 | TC | T | A | 0.011 | 0.035156 | A | T | −0.001226052 |
| rs1421085 | TC | C | T | 0.007454 | 0.035156 | C | T | 0.000830817 |
| rs1424233 | TC | C | T | 0.008826 | 0.035156 | C | T | 0.000983784 |
| rs11957829 | TC | G | A | 0.01121 | 0.035156 | G | A | 0.001249458 |
| rs4129767 | TC | A | G | 0.0145 | 0.035156 | A | G | 0.001616159 |
| rs7134594 | TC | T | C | 0.02048 | 0.035156 | T | C | 0.002282686 |
| rs7560163 | TC | G | C | 0.0213 | 0.035156 | G | C | 0.002374082 |
| rs515135 | TC | T | C | 0.02702 | 0.035156 | T | C | 0.003011629 |
| rs3120140 | TC | A | G | 0.0391 | 0.035156 | A | G | 0.004358057 |
| rs507666 | TC | A | G | 0.06781 | 0.035156 | A | G | 0.007558097 |
| rs6544713 | TC | T | C | 0.075133 | 0.035156 | T | C | 0.00837428 |
| rs2292318 | PP | C | T | 0.009271 | 0.020113 | T | C | −0.001682473 |
| rs10821415 | PP | C | A | 0.005237 | 0.020113 | A | C | −0.000267579 |
| rs2519093 | PP | T | C | 0.00675 | 0.020113 | T | C | 3.8304E−05 |
| rs1333042 | PP | A | G | 0.003109 | 0.020113 | A | G | 0.000564212 |
| rs7916879 | PP | G | A | 0.003229 | 0.020113 | G | A | 0.000585989 |
| rs312949 | PP | C | G | 0.004806 | 0.020113 | C | G | 0.000872178 |
| rs11196288 | PP | G | A | 0.008032 | 0.020113 | G | A | 0.001457623 |
| rs1867624 | PP | C | T | 0.005385 | 0.020113 | C | T | 0.002388245 |
| rs35419456 | PP | A | C | 0.02499 | 0.020113 | A | C | 0.00453511 |
| rs2200733 | AF | T | C | 0.536 | 0.007708 | C | T | −0.005903052 |
| rs11191416 | AF | G | T | 0.058 | 0.007708 | G | T | 0.000638763 |
| rs1200159 | AF | T | C | 0.067 | 0.007708 | T | C | 0.000737881 |
| rs12042319 | AF | A | G | 0.084 | 0.007708 | A | G | 0.000925105 |

### Statistical Analysis

Continuous variables in the baseline characteristics of the study population were expressed as means (standard deviations) and categorical variables were expressed as frequencies (percentages). Study participants were categorized into low (the lowest quintile of metaPRS), intermediate (the 2^{nd}-4^{th} quintile of metaPRS) and high (the highest quintile of metaPRS) genetic risk groups based on metaPRS levels.

A stratified Cox proportional risk regression model with sex-adjusted, age-based time scales was used to calculate genetic risk scores, hazard ratios (HRs) and 95% confidence intervals (CIs) of major clinical risk factors to stroke incidence. Cumulative incidence curves corrected for sex were plotted using “survfit.coxph” (R package “survival”) to assess the lifetime risk of stroke at age 80 in study subjects stratified by different genetic risks and major clinical risk factors. Absolute risk reduction (ARR) was calculated based on the difference in lifetime risk values between the suboptimal and optimal CVH groups, and a weighted least squares regression model was used to assess the increasing trend of ARR with a genetic risk. Bonferroni correction was used to adjust for multiple testing, and differences were considered to reach statistical significance when the two-sided P value <0.007 (P value divided by the number of multiple tests, i.e., 0.05/7). All analysis were performed using the R software version 3.6.0 (R Foundation for Statistical Computing, Vienna, Austria) or the SAS statistical software version 9.4 (SAS Institute Inc, Cary, NC).

Genetic Risk grouping of the Study Population

Table 4 shows the baseline characteristics of the 41,006 study subjects in the cohort population. The mean age of the total population was 51.9 (10.6) years and 43.1% were male. Participants at a high genetic risk (upper 20% in metaPRS) had higher cardiometabolic risk factors (hypertension, diabetes, dyslipidaemia). After 367,750 person-years of follow-up (9.0 mean follow-up years), 1,227 participants had a stroke before the age of 80, including 769 ischaemic strokes, 355 haemorrhagic strokes, 21 ischaemic strokes with haemorrhagic strokes, and 124 strokes of an unspecified subtype.

TABLE 4
Baseline information on prospective cohorts

| | Total | | Genetic risk grouping | | | | | |
|---|---|---|---|---|---|---|---|---|
| Characteristics | (N = 41,006) | | Low (N = 8202) | | Medium (N = 24,603) | | High (N = 8201) | |
| Age, years | 51.9 | (10.6) | 51.6 | (10.4) | 52.0 | (10.6) | 51.7 | (10.5) |
| Male, N (%) | 17,684 | (43.1) | 3510 | (42.8) | 10,653 | (43.3) | 3521 | (42.9) |
| Body mass index, kg/m² | 23.8 | (3.6) | 23.3 | (3.5) | 23.8 | (3.6) | 24.1 | (3.6) |
| Current smoker, N (%) | 10,531 | (26.0) | 2076 | (25.7) | 6369 | (26.2) | 2086 | (25.8) |
| Systolic blood pressure, mmHg | 127.7 | (21.5) | 124.0 | (19.9) | 127.8 | (21.6) | 131.0 | (22.1) |
| Diastolic blood pressure, mmHg | 79.2 | (11.8) | 77.3 | (11.2) | 79.2 | (11.8) | 81.0 | (12.0) |
| Total cholesterol, mg/dl | 180.4 | (36.3) | 178.7 | (36.2) | 180.2 | (36.2) | 182.7 | (36.2) |
| Fasting blood sugar, mg/dl | 94.2 | (26.6) | 92.5 | (24.4) | 94.1 | (26.8) | 96.0 | (28.0) |
| Hypertension, N (%) | 13,382 | (32.6) | 2083 | (25.4) | 8045 | (32.7) | 3254 | (39.7) |
| Diabetes, N (%) | 2416 | (5.9) | 396 | (4.9) | 1428 | (5.8) | 592 | (7.3) |
| Dyslipidemia, N (%) | 13,228 | (32.8) | 2357 | (29.4) | 7945 | (32.8) | 2926 | (36.4) |
| Family history of stroke, N (%) | 2803 | (6.8) | 458 | (5.6) | 1648 | (6.7) | 697 | (8.5) |

Continuous variables are expressed as mean (standard deviation) and categorical variables are expressed as number (percentage).

### Polygenic Risk Score Construction and Stroke Prediction

The optimal stroke sub-phenotype (Stroke) PRS identified a set of stroke risk-related genes associated with East Asian populations, which included 280 Stroke-associated single-nucleotide polymorphism (SNP) sites as shown in Table 3, and the detection of these SNP sites and the determination of genetic risk scores for the incidence risk by Σβi×Ni provided a good evaluation of the risk of stroke incidence in East Asian populations. Here, for the effect values of each Stroke-related SNP, the effect values of the SNPs in the sub-phenotype PRS column in Table 3 could be uniformly used, or the effect values of the SNPs in the metaPRS column in Table 3 could be uniformly used. The higher the genetic risk score, the higher the individual's risk of stroke incidence.

There were varying degrees of correlation between the 14 subphenotypes of PRS (FIG. 3).

In addition to the detection of the 280 Stroke-associated SNPs shown in Table 3, the protocol of the present invention for evaluating the risk of stroke incidence can further selectively detect one or more sets of SNPs among the 159 CAD-associated SNPs, 4 SBP-associated SNPs, 1 WC-associated SNP, 55 T2D-associated SNPs, 22 TC-associated SNPs, 9 PP-associated SNPs, and 4 AF-associated SNPs as shown in Table 3, and obtain a genetic risk score for the risk of morbidity by means of Σβi×Ni, which allows a better evaluation of the risk of stroke incidence in East Asian populations. When the protocol of the present invention for evaluating the risk of stroke incidence comprises the detection of one or more of CAD, SBP, WC, T2D, TC, PP, AF-related SNPs, for the effect values of these SNPs, the effect values of the SNPs in the sub-phenotype PRS column of Table 3 could be used, and the effect values of the SNPs in the metaPRS column of Table 3 are preferably used. The higher the genetic risk score, the higher the individual's risk of stroke incidence.

The metaPRS containing the 534 SNPs shown in Table 3 had a stronger association with stroke than any other subphenotypic PRSs, and for each standard deviation increment in metaPRS, the HRs (95% CI) for total stroke, ischemic stroke, and hemorrhagic stroke were 1.28 (1.21-1.36), 1.29 (1.20-1.39) and 1.30 (1.17-1.45), respectively (FIG. 4). Further adjustment for clinical risk factors including family history of stroke (Table 5) suggests that the metaPRS of the present invention can be used to assess the risk of stroke incidence independently of traditional clinical risk factors.

TABLE 5
Association of metaPRS (with each increment in standard deviation) with
stroke incidence, adjusted or unadjusted for clinical risk factors

| | All stroke | | Ischemic stroke | | Hemorrhagic stroke | |
|---|---|---|---|---|---|---|
| | (N = 1227) | | (N = 769) | | (N = 355) | |
| Model | HR (95% CI) | P value | HR (95% CI) | P value | HR (95% CI) | P value |
| metaPRS | 1.28 (1.21−1.36) | 5.06 × 10⁻¹⁸ | 1.29 (1.20−1.39) | 2.07 × 10⁻¹² | 1.30 (1.17−1.45) | 8.01 × 10⁻⁷ |
| metaPRS + family | 1.27 (1.20−1.35) | 6.46 × 10⁻¹⁷ | 1.28 (1.19−1.37) | 1.54 × 10⁻¹¹ | 1.29 (1.16−1.44) | 1.78 × 10⁻⁶ |
| history of stroke | | | | | | |
| metaPRS + | 1.22 (1.15−1.29) | 7.29 × 10⁻¹² | 1.23 (1.15−1.32) | 1.41 × 10⁻⁸ | 1.23 (1.10−1.37) | 1.49 × 10⁻⁴ |
| hypertension | | | | | | |
| metaPRS + diabetes | 1.28 (1.21−1.35) | 3.20 × 10⁻¹⁷ | 1.28 (1.19−1.38) | 8.36 × 10⁻¹² | 1.30 (1.17−1.44) | 1.31 × 10⁻⁶ |
| metaPRS + | 1.27 (1.20−1.34) | 2.56 × 10⁻¹⁶ | 1.27 (1.19−1.37) | 3.93 × 10⁻¹¹ | 1.29 (1.16−1.44) | 2.32 × 10⁻⁶ |
| dyslipidemia | | | | | | |
| metaPRS + body | 1.27 (1.20−1.34) | 1.60 × 10⁻¹⁶ | 1.27 (1.19−1.37) | 3.31 × 10⁻¹¹ | 1.29 (1.16−1.44) | 1.75 × 10⁻⁶ |
| mass index | | | | | | |
| metaPRS + 5 | 1.21 (1.14−1.28) | 8.57 × 10⁻¹¹ | 1.21 (1.13−1.30) | 1.92 × 10⁻⁷ | 1.22 (1.10−1.36) | 2.29 × 10⁻⁴ |
| clinical risk factors | | | | | | |

Hazard ratios (HR) and 95% confidence intervals (CI) were calculated using cohort-stratified, age-scaled Cox proportional risk regression models, adjusted for sex, and adjusted or unadjusted for clinical risk factors.

In the present invention, metaPRS genetic risk stratification was performed based on the total population metaPRS genetic risk score (Table 6). Individuals with a metaPRS genetic risk score <−0.140 were determined to be at a low genetic risk of stroke incidence (metaPRS 0 to 20%), and individuals with a metaPRS genetic risk score >0.305 were determined to be at a high genetic risk of stroke incidence (metaPRS 80 to 100%).

TABLE 6
MetaPRS genetic risk stratification quick reference table

| | Group | | | | |
|---|---|---|---|---|---|
| | 0-20% | 20%-40% | 40%-60% | 60%-80% | 80%-100% |
| | (low) | (medium) | (medium) | (medium) | (high) |
| Genetic | <−0.140 | −0.140~0.019 | 0.019~0.154 | 0.154~0.305 | >0.305 |
| risk score | | | | | |

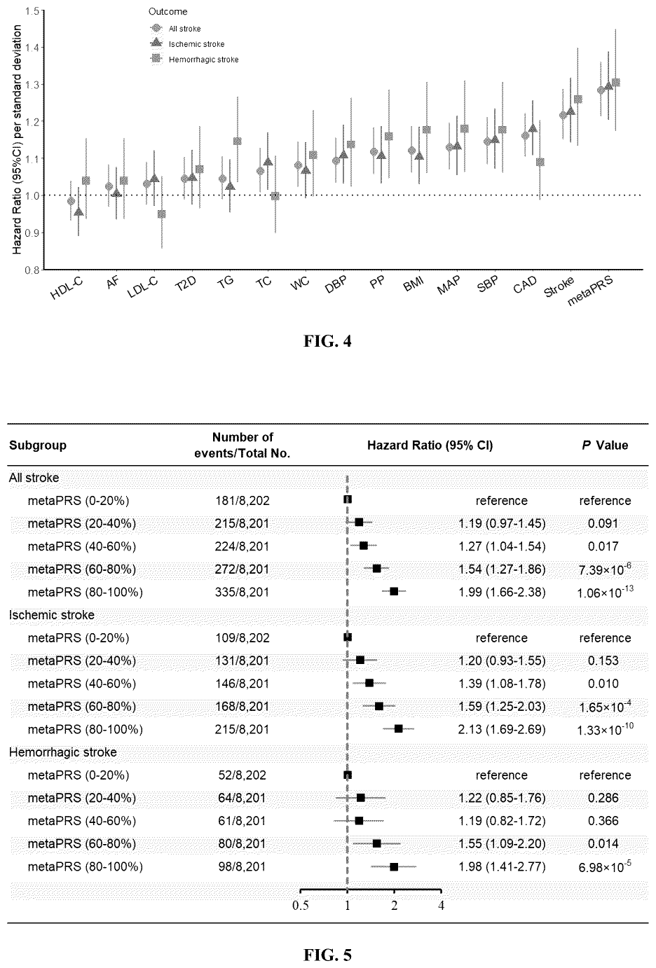

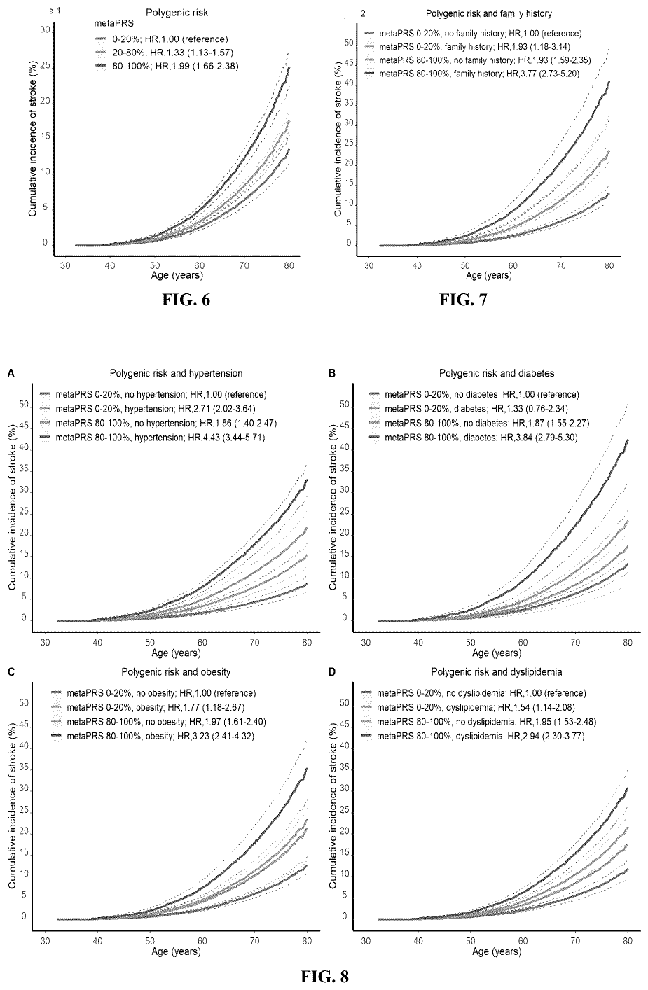

After grouping the population according to the 5 quintiles of metaPRS, the groups showed a clear gradient in stroke risk (trend P value <0.001) (FIG. 5). Compared with those at a low genetic risk (lower 20% in metaPRS), those at a high genetic risk (upper 20% in metaPRS) had an approximately 2-fold higher risk of stroke (HR: 1.99, 95% CI: 1.66-2.38, P=1.11×10⁻¹³) (FIG. 6). The lifetime risk of stroke (risk of stroke at age 80) was also nearly two times higher in the individuals with a high genetic risk than in those with a low genetic risk (25.2%, 95% CI: 22.5%-27.7%, and 13.6%, 95% CI: 11.6%-15.5%, respectively).

### Lifetime Risk of Stroke by Combined Genetic Risk and Major Risk Factor Stratification

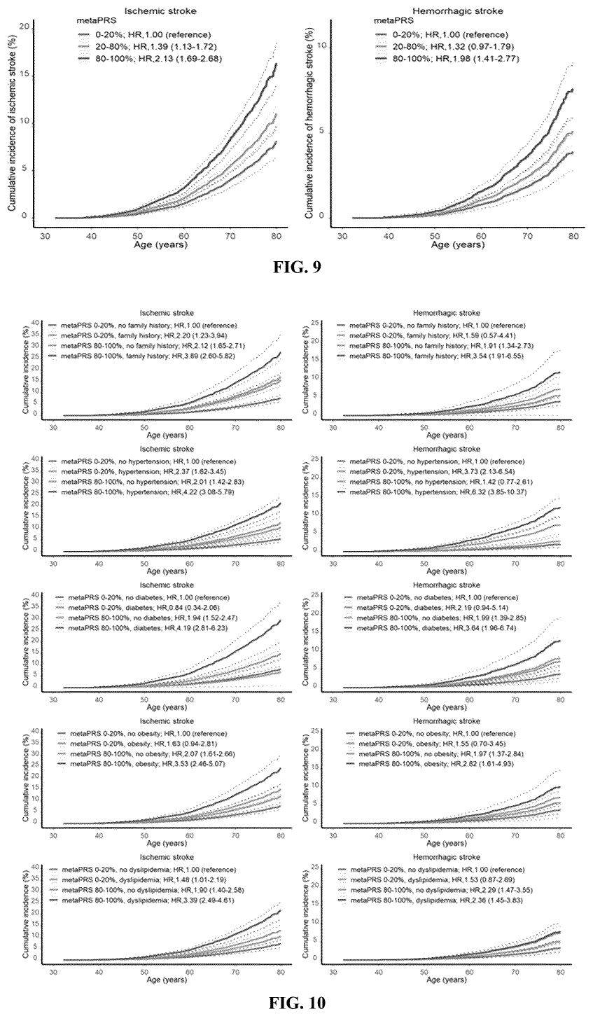

There were significant differences in lifetime risk of stroke under different genetic risk and major clinical risk factor stratifications (FIGS. 7 and 8). For example, individuals with a low genetic risk and no family history of stroke had a lifetime risk of stroke of 13.2% (95% CI: 11.1%-15.1%), whereas individuals with either risk factor of a high genetic risk and a family history of stroke had nearly the same lifetime risk of stroke (23.9%, 95% CI: 21.1%-26.5%, and 23.7%, 95% CI. 13.4%-32.8%); and when they had both, the lifetime risk of stroke could be as high as 41.1% (95% CI: 31.4%-49.5%). A similar gradient of lifetime risk of stroke was observed in the stratification of genetic risk and the other four clinical risk factors (hypertension, diabetes, dyslipidaemia, obesity) (FIG. 8, Table 7).

The above genetic risk outcomes or risk outcomes after combining the major risk factors were similar in terms of effect and risk for haemorrhagic and ischaemic stroke (FIGS. 9 and 10).

TABLE 7
Lifetime risk of stroke combining genetic and clinical risk factors

| | | | Genetic risk stratification | | | |
|---|---|---|---|---|---|---|
| Lifetime risk of stroke (%) | | | low | | medium | high |
| Clinical | family history | No | 13.2 | (11.1-15.1) | 17.1 (15.6-18.6) | 23.9 (21.1-26.5) |
| risk | of stroke | Yes | 23.7 | (13.4-32.8) | 27.2 (21.5-32.6) | 41.1 (31.4-49.5) |
| factors | hypertension | No | 8.7 | (6.8-10.5) | 11.1 (9.7-12.4) | 15.5 (12.7-18.2) |
| | | Yes | 21.9 | (17.9-25.7) | 25.2 (23.0-27.5) | 33.2 (29.3-36.8) |
| | diabetes | No | 13.4 | (11.3-15.3) | 16.8 (15.3-18.2) | 23.5 (20.8-26.1) |
| | | Yes | 17.6 | (8.4-25.8) | 27.5 (21.9-32.7) | 42.5 (32.5-50.9) |
| | dyslipidemia | No | 11.8 | (9.6-14.0) | 14.7 (13.2-16.3) | 21.7 (18.6-24.6) |
| | | Yes | 17.7 | (13.8-21.4) | 22.9 (20.4-25.3) | 30.9 (26.5-35.1) |
| | obesity | No | 12.8 | (10.7-14.7) | 16.3 (14.9-17.8) | 23.5 (20.7-26.1) |
| | | Yes | 21.4 | (13.8-28.2) | 26.3 (22.2-30.2) | 35.5 (28.0-42.2) |

### Example 2

Practical Application Case 1: The individual to be evaluated, Li, a Chinese Han, female, 35 years old, with a combined family history of stroke, was evaluated for a high or low genetic risk of stroke incidence using the detection device of the present invention for evaluating the genetic risk of stroke, and was given guidance advice in combination with traditional risk factors. The following procedure was carried out: fasting blood was collected, DNA was isolated from the anticoagulated blood of the individual to be evaluated, and genotypes at 534 sites were assayed using the Illumina Hiseq X Ten sequencer.

The genotypes of the 534 sites tested for Li are shown in Table 8:

TABLE 8

| | | Number of | | | Number of | | | Number of | | | Number of |
|---|---|---|---|---|---|---|---|---|---|---|---|
| | Geno- | effector | | Geno- | effector | | Geno- | effector | | Geno- | effector |
| SNP | types | alleles | SNP | types | alleles | SNP | types | alleles | SNP | types | alleles |
| rs10010670 | GA | 1 | rs174546 | TC | 1 | rs4932370 | AG | 1 | rs16927668 | CT | 1 |
| rs10051787 | TC | 1 | rs174547 | CT | 1 | rs4939883 | TC | 1 | rs16933812 | GT | 1 |
| rs10064156 | TT | 0 | rs17465637 | CC | 0 | rs499974 | CC | 0 | rs16967013 | CC | 0 |
| rs10093110 | GG | 0 | rs17477177 | CT | 1 | rs507666 | GG | 0 | rs16986953 | AG | 1 |
| rs10096633 | TC | 1 | rs17514846 | AC | 1 | rs515135 | CC | 0 | rs16990971 | AA | 0 |
| rs10139550 | GC | 1 | rs17517928 | CC | 0 | rs5215 | CT | 1 | rs16999793 | GG | 0 |
| rs10160804 | AA | 2 | rs17581137 | AA | 0 | rs556621 | TG | 1 | rs17030613 | CC | 2 |
| rs10203174 | CC | 0 | rs17608766 | TT | 0 | rs55783344 | CC | 0 | rs17080091 | TC | 1 |
| rs10237377 | GG | 2 | rs17609940 | GG | 0 | rs56062135 | CC | 0 | rs17080102 | CG | 1 |
| rs10260816 | GG | 0 | rs17612742 | CT | 1 | rs56289821 | GG | 0 | rs17087335 | TG | 1 |
| rs10267593 | GG | 0 | rs17678683 | TT | 0 | rs56336142 | TT | 0 | rs17122278 | GA | 1 |
| rs1027087 | TT | 2 | rs17680741 | TT | 0 | rs574367 | GG | 0 | rs17135399 | AA | 0 |
| rs10278336 | GA | 1 | rs17695224 | AG | 1 | rs579459 | TT | 0 | rs17150703 | GG | 0 |
| rs1029420 | CT | 1 | rs17791513 | GA | 1 | rs582384 | CC | 0 | rs17249754 | GG | 0 |
| rs1037814 | TC | 1 | rs17843768 | CC | 0 | rs58542926 | CC | 0 | rs17301514 | GG | 0 |
| rs10401969 | TT | 0 | rs17843797 | TT | 0 | rs590121 | GG | 0 | rs173396 | AG | 1 |
| rs10455782 | TC | 1 | rs1799945 | CC | 0 | rs5996074 | GA | 1 | rs17358402 | CC | 0 |
| rs10507248 | GG | 0 | rs1800234 | TT | 0 | rs60154123 | TC | 1 | rs17381664 | TT | 0 |
| rs10512861 | GG | 0 | rs1800588 | CC | 0 | rs6038557 | GA | 1 | rs4731420 | GG | 0 |
| rs10513801 | TT | 0 | rs1801282 | GC | 1 | rs6065311 | CC | 0 | rs4735692 | AA | 2 |
| rs1052053 | GA | 1 | rs180327 | TT | 0 | rs6093446 | AG | 1 | rs4752700 | GG | 2 |
| rs10745332 | AA | 0 | rs1805081 | CT | 1 | rs61776719 | AC | 1 | rs4757391 | TT | 0 |
| rs10757274 | GA | 1 | rs181359 | GG | 0 | rs633185 | GG | 0 | rs4766228 | GG | 0 |
| rs10773003 | GG | 0 | rs181360 | TT | 0 | rs634501 | GG | 0 | rs4776970 | TT | 0 |
| rs1077834 | TT | 0 | rs1832007 | GA | 1 | rs6490029 | AA | 0 | rs4788102 | GG | 0 |
| rs10820405 | GG | 0 | rs1861411 | GG | 0 | rs6494488 | AA | 0 | rs4812829 | AA | 2 |
| rs10821415 | CC | 0 | rs1867624 | CT | 1 | rs651821 | TT | 0 | rs4821382 | CC | 0 |
| rs10824026 | GG | 2 | rs1868673 | CC | 0 | rs6544713 | CC | 0 | rs4836831 | CT | 1 |
| rs10830963 | GG | 2 | rs1870634 | TG | 1 | rs6545814 | AA | 0 | rs4845625 | TC | 1 |
| rs10842992 | TT | 0 | rs1883025 | TC | 1 | rs660599 | GG | 0 | rs4846049 | GG | 0 |
| rs10857147 | TA | 1 | rs1887320 | GG | 2 | rs663129 | AA | 2 | rs4883201 | GA | 1 |
| rs10886471 | TC | 1 | rs1892094 | CC | 0 | rs6666258 | GG | 0 | rs4883263 | CC | 0 |
| rs10889353 | AA | 0 | rs1902859 | CT | 1 | rs667920 | TT | 0 | rs4911495 | AA | 0 |
| rs10923931 | GG | 0 | rs191835914 | AA | 0 | rs6700559 | CT | 1 | rs4917014 | GT | 1 |
| rs10953541 | CC | 0 | rs1976041 | AA | 2 | rs671 | GG | 0 | rs4918072 | AG | 1 |
| rs10968576 | AA | 0 | rs1982963 | AA | 0 | rs67156297 | GG | 0 | rs4923678 | AA | 0 |
| rs11030104 | GA | 1 | rs2000813 | CC | 0 | rs67180937 | GG | 0 | rs9512699 | AA | 0 |
| rs11057830 | GG | 0 | rs2000999 | AG | 1 | rs6725887 | TT | 0 | rs9534262 | TC | 1 |
| rs11066280 | TT | 0 | rs200990725 | CC | 0 | rs67839313 | TT | 0 | rs9552911 | GG | 0 |
| rs11067763 | GA | 1 | rs2021783 | TC | 1 | rs6795735 | TT | 0 | rs9568867 | GG | 0 |
| rs11077501 | TT | 0 | rs2028299 | AA | 0 | rs6807945 | TT | 0 | rs9591012 | GG | 0 |
| rs11099493 | AA | 0 | rs2043085 | TC | 1 | rs6808574 | CC | 0 | rs9593 | TA | 1 |
| rs11125936 | CT | 1 | rs2057291 | GG | 0 | rs6813195 | TC | 1 | rs964184 | CC | 0 |
| rs11136341 | AA | 0 | rs2066714 | TC | 1 | rs6817105 | TC | 1 | rs9663362 | CC | 0 |
| rs11142387 | CA | 1 | rs2068888 | AA | 0 | rs6818397 | TG | 1 | rs9687065 | AA | 0 |
| rs1116357 | GA | 1 | rs2074158 | TT | 0 | rs6825454 | TT | 0 | rs975722 | AA | 0 |
| rs11191416 | GT | 1 | rs2075260 | GA | 1 | rs6825911 | CT | 1 | rs9810888 | GG | 2 |
| rs11196288 | GA | 1 | rs2075291 | CC | 0 | rs6829822 | TG | 1 | rs9815354 | GG | 0 |
| rs11205760 | TT | 0 | rs2075423 | TG | 1 | rs6831256 | GA | 1 | rs9818870 | CC | 0 |
| rs11206510 | TT | 0 | rs2081687 | CC | 0 | rs6838973 | CT | 1 | rs9828933 | CC | 2 |
| rs11222084 | AA | 0 | rs2106261 | CC | 0 | rs6871667 | GA | 1 | rs984222 | CG | 1 |
| rs11257655 | TT | 0 | rs2107595 | AA | 2 | rs6878122 | AA | 0 | rs9892152 | TC | 1 |
| rs1129555 | GG | 0 | rs2123536 | TC | 1 | rs6882076 | CC | 0 | rs995000 | CC | 0 |
| rs11509880 | AA | 0 | rs2128739 | CA | 1 | rs6905288 | GA | 1 | rs9970807 | CC | 0 |
| rs1152591 | GG | 0 | rs2144300 | CC | 0 | rs6909752 | AG | 1 | rs1514175 | AA | 0 |
| rs11556924 | CC | 0 | rs2145598 | GG | 0 | rs6960043 | CC | 2 | rs1532085 | AG | 1 |
| rs11557092 | TC | 1 | rs2156552 | AT | 1 | rs6984210 | CC | 0 | rs1535500 | GG | 0 |
| rs11601507 | CC | 0 | rs216172 | CG | 1 | rs699 | GG | 0 | rs1552224 | AA | 0 |
| rs11604680 | GA | 1 | rs2200733 | CT | 1 | rs6997340 | CT | 1 | rs1555543 | CC | 0 |
| rs11624704 | AA | 0 | rs2213732 | AA | 0 | rs702485 | GG | 0 | rs1558902 | TT | 0 |
| rs11634397 | AA | 0 | rs2229383 | TT | 0 | rs702634 | GA | 1 | rs1575972 | TT | 0 |
| rs11651052 | AA | 2 | rs2230808 | CC | 0 | rs7087591 | GA | 1 | rs1591805 | GG | 0 |
| rs11660468 | TC | 1 | rs2237892 | CC | 0 | rs7107784 | AA | 0 | rs16844401 | GG | 0 |
| rs11677932 | GG | 0 | rs2237896 | GG | 0 | rs7116641 | TT | 0 | rs16849225 | CC | 0 |
| rs1169288 | CA | 1 | rs2240736 | CC | 0 | rs7134594 | TC | 1 | rs16858082 | CC | 0 |
| rs1173766 | CC | 0 | rs2245019 | AA | 0 | rs7136259 | CC | 0 | rs16896398 | TA | 1 |
| rs117601636 | AA | 0 | rs2258287 | AA | 0 | rs7164883 | AA | 0 | rs1689800 | AA | 0 |
| rs117711462 | GG | 0 | rs2261181 | TC | 1 | rs7178572 | GA | 1 | rs4409766 | CT | 1 |
| rs11787792 | AA | 0 | rs2292318 | CC | 0 | rs7185272 | GC | 1 | rs4420638 | AA | 0 |
| rs11810571 | GC | 1 | rs2295786 | AT | 1 | rs7193343 | CT | 1 | rs4458523 | GG | 0 |
| rs11830157 | TT | 0 | rs2296172 | AA | 0 | rs7199941 | AG | 1 | rs4468572 | TC | 1 |
| rs11838267 | TT | 0 | rs2297991 | CC | 0 | rs7202877 | TT | 0 | rs4471613 | GG | 0 |
| rs11838776 | GG | 0 | rs2302593 | CC | 0 | rs7206541 | TT | 0 | rs459193 | AG | 1 |
| rs11847697 | CC | 0 | rs2303790 | AA | 0 | rs7208487 | GT | 1 | rs4593108 | CC | 0 |
| rs11869286 | GG | 0 | rs2328223 | AA | 0 | rs7213603 | TT | 0 | rs4613862 | AA | 0 |
| rs11957829 | GA | 1 | rs2334499 | CT | 1 | rs7225581 | TT | 0 | rs46522 | TT | 0 |
| rs1200159 | CC | 0 | rs2383208 | AA | 0 | rs7258189 | TT | 0 | rs4713766 | CC | 0 |
| rs12027135 | AA | 0 | rs2415317 | GG | 0 | rs7258445 | AG | 1 | rs4719841 | AG | 1 |
| rs12037987 | CT | 1 | rs243019 | CC | 0 | rs7258950 | AG | 1 | rs4722766 | CG | 1 |
| rs12042319 | GG | 0 | rs246600 | CC | 0 | rs72654473 | CC | 0 | rs4724806 | CC | 0 |
| rs1211166 | AA | 0 | rs247616 | CC | 0 | rs72689147 | GG | 0 | rs9309245 | GC | 1 |
| rs12202017 | AA | 0 | rs2487928 | GG | 0 | rs73015714 | CC | 0 | rs93138 | GT | 1 |
| rs12204590 | TT | 0 | rs2519093 | CC | 0 | rs7304841 | CA | 1 | rs9319428 | AG | 1 |
| rs12214416 | TT | 0 | rs2531995 | TC | 1 | rs7306455 | GG | 0 | rs9349379 | GG | 0 |
| rs12229654 | TT | 0 | rs2535633 | GG | 2 | rs7306523 | GA | 1 | rs9357121 | TT | 0 |
| rs12242953 | GG | 0 | rs2571445 | AG | 1 | rs73069940 | CC | 0 | rs9367716 | TT | 0 |
| rs12415501 | CC | 0 | rs2575876 | AG | 1 | rs736699 | GA | 1 | rs9376090 | CT | 1 |
| rs12438008 | AG | 1 | rs261967 | CA | 1 | rs737337 | TT | 0 | rs9390698 | AG | 1 |
| rs12445022 | GG | 0 | rs2625967 | AA | 0 | rs738409 | GC | 1 | rs944172 | CT | 1 |
| rs12453914 | CC | 0 | rs2642442 | TT | 0 | rs7403531 | CC | 0 | rs9470794 | TT | 0 |
| rs12463617 | CC | 0 | rs273909 | AA | 0 | rs740406 | AA | 0 | rs9473924 | TG | 1 |
| rs12500824 | AA | 2 | rs2758607 | AG | 1 | rs7405452 | CC | 0 | rs9501744 | CC | 0 |
| rs1250229 | CC | 0 | rs2782980 | TC | 1 | rs748431 | TT | 2 | rs9505118 | GA | 1 |
| rs12524865 | CC | 0 | rs2783963 | AG | 1 | rs7499892 | TT | 2 | rs1421085 | TT | 0 |
| rs12535846 | AA | 2 | rs2796441 | GG | 2 | rs7500448 | GA | 1 | rs1424233 | CT | 1 |
| rs12549902 | AA | 2 | rs2815752 | AA | 0 | rs7503807 | AA | 0 | rs1436953 | CC | 0 |
| rs12571751 | GG | 2 | rs2819348 | TT | 0 | rs751984 | TT | 0 | rs1448818 | AA | 0 |
| rs12581963 | GG | 0 | rs2820315 | CC | 0 | rs7525649 | CT | 1 | rs1467605 | CC | 0 |
| rs12597579 | CC | 0 | rs2820443 | TT | 0 | rs7528419 | AA | 0 | rs1470579 | CC | 2 |
| rs1260326 | TT | 0 | rs2861568 | AA | 0 | rs7560163 | GC | 1 | rs1495741 | GG | 0 |
| rs12679556 | GG | 0 | rs2925979 | TC | 1 | rs7568458 | TT | 0 | rs1496653 | GA | 1 |
| rs12692735 | GG | 0 | rs2954029 | AT | 1 | rs7610618 | CC | 0 | rs1508798 | TT | 0 |
| rs12718465 | CC | 0 | rs2972143 | GG | 0 | rs7616006 | AA | 0 | rs151193009 | CC | 0 |
| rs12740374 | GG | 0 | rs2972146 | TT | 0 | rs7617773 | CC | 2 | rs4129767 | GG | 0 |
| rs1275988 | TC | 1 | rs29941 | GA | 1 | rs7633770 | AG | 1 | rs4142995 | TT | 0 |
| rs12801636 | AG | 1 | rs3120140 | GG | 0 | rs7678555 | AA | 0 | rs4148008 | CC | 0 |
| rs12897 | AG | 1 | rs312949 | GG | 0 | rs769449 | GG | 0 | rs42039 | CC | 0 |
| rs12927205 | GA | 1 | rs3129853 | GG | 0 | rs76954792 | CC | 0 | rs4266144 | CC | 2 |
| rs12932445 | TT | 0 | rs3130501 | AG | 1 | rs7696431 | TT | 2 | rs4275659 | TT | 2 |
| rs12936587 | GG | 0 | rs3184504 | CC | 0 | rs7701094 | CG | 1 | rs4302748 | AG | 1 |
| rs12946454 | AA | 0 | rs3213545 | AG | 1 | rs7770628 | CT | 1 | rs4377290 | TT | 0 |
| rs12970066 | CC | 0 | rs326214 | AG | 1 | rs780094 | TT | 0 | rs439401 | CC | 2 |
| rs12999907 | AA | 0 | rs34008534 | AA | 0 | rs7810507 | GG | 0 | rs4400058 | GG | 0 |
| rs130071 | GG | 0 | rs340874 | CT | 1 | rs78169666 | AA | 0 | rs871606 | TT | 0 |
| rs13041126 | CT | 1 | rs351855 | AG | 1 | rs7859727 | TT | 0 | rs880315 | TC | 1 |
| rs13078807 | AA | 0 | rs35332062 | GG | 0 | rs7897379 | CT | 1 | rs884366 | GG | 0 |
| rs13115759 | AT | 1 | rs35337492 | AG | 1 | rs7901016 | TT | 0 | rs885150 | CT | 1 |
| rs13143308 | GT | 1 | rs35419456 | CC | 0 | rs7903146 | CC | 0 | rs888789 | AG | 1 |
| rs13143871 | TT | 0 | rs35444 | GA | 1 | rs7916879 | AA | 0 | rs896854 | CC | 0 |
| rs1317507 | AC | 1 | rs36096196 | CC | 0 | rs7917772 | GG | 0 | rs897057 | CC | 0 |
| rs13209747 | TC | 1 | rs368123 | AA | 0 | rs79223353 | GG | 0 | rs9266359 | TC | 1 |
| rs1321309 | GG | 0 | rs376563 | TC | 1 | rs7947761 | AA | 0 | rs9268402 | GG | 0 |
| rs13216675 | CC | 2 | rs3775058 | TA | 1 | rs79548680 | GG | 2 | rs9299 | TT | 0 |
| rs13233731 | AA | 2 | rs3785100 | TT | 0 | rs7955901 | TC | 1 | rs13723 | GA | 1 |
| rs13266634 | TC | 1 | rs3791679 | AG | 1 | rs7965082 | TC | 1 | rs1378942 | CC | 0 |
| rs13277801 | TT | 0 | rs3807989 | AA | 2 | rs7980458 | TT | 0 | rs1412444 | TC | 1 |
| rs13306194 | GG | 0 | rs3809128 | CC | 0 | rs7989336 | AG | 1 | rs3918226 | CC | 0 |
| rs1333042 | GG | 0 | rs3810291 | GG | 0 | rs80234489 | CA | 1 | rs3936511 | AA | 0 |
| rs13342232 | AA | 0 | rs3827066 | CC | 0 | rs8030379 | GA | 1 | rs3993105 | CT | 1 |
| rs1334576 | GA | 1 | rs3846663 | CC | 2 | rs8042271 | GG | 0 | rs838880 | TT | 2 |
| rs13359291 | GA | 1 | rs3861086 | TT | 2 | rs806215 | TC | 1 | rs840616 | CC | 0 |
| rs1344653 | AA | 0 | rs3887137 | TC | 1 | rs8090011 | CG | 1 | rs867186 | GA | 1 |
| rs1359790 | GG | 0 | rs3903239 | GG | 0 | rs8108269 | GG | 0 | rs820430 | AG | 1 |
| rs1367117 | GG | 0 | rs391300 | TC | 1 | | | | | | |

Analysis and processing of the results: the results of the 534 SNPs were compared with Table 3 to find out the genetic contribution of the corresponding effect allele at each site, and weighted and summed to obtain a genetic risk score: genetic risk score=Σβi×Ni, where Bi refers to the effect value of the i^{th} SNP, and Ni refers to the number of effect alleles of the i^{th} SNP carried by the individual.

Evaluation of Li's genetic risk of stroke: Li's genetic risk score for stroke was 0.660, which put her in the high genetic risk group by referring to Table 6. Combined with the fact that Li had a family history of stroke, Li's lifetime risk of stroke was 41.1% by referring to Table 7, which put her in the high-risk group. The combination of genetic and clinical factors predicted that Li had a high risk of stroke, and she was advised to pay further attention to controlling blood pressure, blood glucose, blood lipids and body weight in addition to adopting a healthy lifestyle, to have regular health check-ups, and to consult a doctor in case of any abnormality.

### Modification of the Application Case:

If the individual to be evaluated in the aforementioned Application Case 1 also had high blood pressure, with reference to Table 7, the lifetime risk of stroke was 33.2%, which put her in the high-risk group. It was recommended that she focuses on the intervention and management of blood pressure to reduce the risk of stroke in addition to adopting a healthy lifestyle.

If the individual to be evaluated in the aforementioned Application Case 1 also had diabetes, with reference to Table 7, the lifetime risk of stroke was 42.5%, which put her in the high-risk group. It was recommended that she focuses on the intervention and management of blood glucose to reduce the risk of stroke in addition to adopting a healthy lifestyle.

If the individual to be evaluated in the aforementioned Application Case 1 also had dyslipidemia, with reference to Table 7, the lifetime risk of stroke was 30.9%, which put her in the high-risk group. It was recommended that she focuses on the intervention and management of blood lipid to reduce the risk of stroke in addition to adopting a healthy lifestyle.

If the individual to be evaluated in the aforementioned Application Case 1 also had obesity, with reference to Table 7, the lifetime risk of stroke was 35.5%, which put her in the high-risk group. It was recommended to focus on the intervention and management of body weight by increasing physical activity, balancing dietary nutrition, and reducing fat and high-calorie diets to reduce the risk of stroke.

The individual to be evaluated in the aforementioned Application Case 1 can also be evaluated for the risk of stroke incidence of the individual by obtaining a genetic risk score for morbidity risk by Σβi×Ni based on the results of the 280 Stroke-associated SNPs tested in Table 8, or by further combining the results of the 159 CAD-associated SNPs, 4 SBP-associated SNPs, 1 WC-associated SNP and/or 55 T2D-associated SNPs shown in Table 8, or by even further combining the results of the 22 TC-associated SNPs, 9 PP-associated SNPs, 4 AF-associated SNPs.

1. A method for evaluating a risk of stroke incidence, comprising:
detecting a sample from an individual to obtain the individual's information, wherein the individual information comprises the following single nucleotide polymorphism site information:
stroke-related single nucleotide polymorphism sites: rs10051787, rs10093110, rs10139550, rs10160804, rs10237377, rs10260816, rs10267593, rs10278336, rs1037814, rs10507248, rs10512861, rs10745332, rs10757274, rs10773003, rs10824026, rs10857147, rs10953541, rs10968576, rs11099493, rs1116357, rs11206510, rs11222084, rs11257655, rs11509880, rs1152591, rs11557092, rs11601507, rs11604680, rs11624704, rs11677932, rs1173766, rs117601636, rs117711462, rs11787792, rs11810571, rs11838776, rs11869286, rs12027135, rs12037987, rs12202017, rs12229654, rs12415501, rs12438008, rs12445022, rs12500824, rs1250229, rs12549902, rs12571751, rs12581963, rs12692735, rs12718465, rs12801636, rs12897, rs12927205, rs12932445, rs12936587, rs12946454, rs13143308, rs13209747, rs1321309, rs13216675, rs13233731, rs13342232, rs1334576, rs13359291, rs1344653, rs1359790, rs1367117, rs13723, rs1412444, rs1436953, rs1470579, rs1495741, rs1508798, rs151193009, rs1552224, rs1591805, rs16844401, rs16849225, rs16858082, rs16896398, rs16967013, rs16999793, rs17030613, rs17080091, rs17087335, rs17122278, rs17135399, rs17301514, rs173396, rs17358402, rs17477177, rs17514846, rs17581137, rs17612742, rs17680741, rs17791513, rs180327, rs181359, rs1861411, rs1868673, rs1870634, rs1887320, rs1892094, rs1902859, rs191835914, rs1976041, rs1982963, rs2000813, rs2028299, rs2057291, rs2068888, rs2074158, rs2075291, rs2075423, rs2107595, rs2128739, rs2145598, rs216172, rs2213732, rs2229383, rs2237896, rs2240736, rs2245019, rs2261181, rs2295786, rs2334499, rs243019, rs246600, rs247616, rs2487928, rs2535633, rs2575876, rs261967, rs273909, rs2758607, rs2782980, rs2796441, rs2815752, rs2820315, rs2861568, rs2925979, rs2972146, rs29941, rs326214, rs340874, rs351855, rs35337492, rs35444, rs36096196, rs368123, rs376563, rs3775058, rs3785100, rs3791679, rs3861086, rs3887137, rs3903239, rs3936511, rs4275659, rs4400058, rs4409766, rs4458523, rs4468572, rs4593108, rs46522, rs4719841, rs4722766, rs4724806, rs4731420, rs4752700, rs4766228, rs4788102, rs4812829, rs4821382, rs4836831, rs4846049, rs4883263, rs4911495, rs4918072, rs4932370, rs556621, rs56062135, rs574367, rs579459, rs582384, rs5996074, rs6093446, rs61776719, rs633185, rs6490029, rs6545814, rs663129, rs6666258, rs667920, rs6700559, rs671, rs67156297, rs67180937, rs6725887, rs67839313, rs6795735, rs6813195, rs6817105, rs6825454, rs6825911, rs6829822, rs6831256, rs6838973, rs6878122, rs6882076, rs6905288, rs6909752, rs6960043, rs699, rs6997340, rs702485, rs702634, rs7136259, rs7164883, rs7178572, rs7193343, rs7199941, rs7202877, rs7206541, rs7258189, rs7258445, rs7258950, rs72689147, rs73015714, rs7304841, rs7306455, rs73069940, rs736699, rs737337, rs7403531, rs740406, rs7499892, rs7500448, rs7503807, rs7568458, rs7610618, rs7616006, rs7696431, rs7770628, rs780094, rs7810507, rs7859727, rs7917772, rs79223353, rs7947761, rs7955901, rs7965082, rs7980458, rs8042271, rs8108269, rs838880, rs840616, rs871606, rs880315, rs884366, rs885150, rs888789, rs9266359, rs9268402, rs9299, rs9319428, rs9376090, rs9473924, rs9505118, rs9568867, rs964184, rs9687065, rs975722, rs9810888, rs9815354, rs9828933, rs984222, rs9892152, rs9970807.

2. The method according to claim 1, wherein the individual information further comprises the following single nucleotide polymorphism site information:
CAD-related single nucleotide polymorphism sites: rs10096633, rs10203174, rs1027087, rs1029420, rs10401969, rs10455782, rs10513801, rs1077834, rs10820405, rs10830963, rs10842992, rs10886471, rs11030104, rs11057830, rs11066280, rs11067763, rs11077501, rs11125936, rs11136341, rs11142387, rs11205760, rs1129555, rs11556924, rs11634397, rs1169288, rs11830157, rs11838267, rs11847697, rs1211166, rs12204590, rs12214416, rs12242953, rs12453914, rs12463617, rs12524865, rs12535846, rs12597579, rs12679556, rs12740374, rs12970066, rs12999907, rs130071, rs13041126, rs13078807, rs1317507, rs13266634, rs13277801, rs13306194, rs1378942, rs1467605, rs1496653, rs1514175, rs1535500, rs1555543, rs1558902, rs1575972, rs1689800, rs16933812, rs16986953, rs16990971, rs17080102, rs17150703, rs17249754, rs17381664, rs174547, rs17465637, rs17517928, rs17609940, rs17678683, rs17695224, rs17843768, rs1799945, rs1800234, rs1801282, rs181360, rs2000999, rs200990725, rs2021783, rs2043085, rs2066714, rs2075260, rs2106261, rs2144300, rs2237892, rs2296172, rs2302593, rs2328223, rs2383208, rs2415317, rs2531995, rs2571445, rs2642442, rs2819348, rs2820443, rs3129853, rs3130501, rs3213545, rs35332062, rs3809128, rs3827066, rs3846663, rs391300, rs3993105, rs4148008, rs4266144, rs4377290, rs439401, rs4420638, rs4471613, rs459193, rs4613862, rs4713766, rs4735692, rs4757391, rs4845625, rs4917014, rs4923678, rs499974, rs5215, rs55783344, rs56289821, rs56336142, rs590121, rs6065311, rs6494488, rs651821, rs660599, rs6807945, rs6808574, rs6818397, rs7087591, rs7107784, rs7116641, rs7225581, rs72654473, rs748431, rs7525649, rs7617773, rs78169666, rs7901016, rs7989336, rs8030379, rs8090011, rs820430, rs867186, rs896854, rs897057, rs9309245, rs93138, rs9349379, rs9357121, rs9367716, rs9390698, rs944172, rs9470794, rs9534262, rs9552911, rs9593, rs995000;
SBP-related single nucleotide polymorphism sites: rs1275988, rs7701094, rs7405452, rs751984;
WC-related single nucleotide polymorphism site: rs2303790; and
T2D-related single nucleotide polymorphism sites: rs10010670, rs10064156, rs1052053, rs10923931, rs11651052, rs11660468, rs1260326, rs13143871, rs1448818, rs1532085, rs16927668, rs174546, rs17608766, rs17843797, rs1800588, rs1832007, rs2081687, rs2123536, rs2156552, rs2230808, rs2258287, rs2297991, rs2783963, rs2954029, rs3807989, rs3810291, rs3918226, rs4142995, rs42039, rs4302748, rs4776970, rs4883201, rs58542926, rs60154123, rs6038557, rs634501, rs6871667, rs6984210, rs7185272, rs7208487, rs7213603, rs738409, rs7528419, rs7678555, rs769449, rs76954792, rs7897379, rs7903146, rs79548680, rs80234489, rs806215, rs9501744, rs9512699, rs9591012, rs9818870;
preferably, the individual information further comprises the following single nucleotide polymorphism site information:
TC-related single nucleotide polymorphism sites: rs10889353, rs11957829, rs13115759, rs1421085, rs1424233, rs1805081, rs1883025, rs2625967, rs2972143, rs3120140, rs3184504, rs34008534, rs4129767, rs4939883, rs507666, rs515135, rs6544713, rs7134594, rs7306523, rs7560163, rs7633770, rs9663362;
PP-related single nucleotide polymorphism sites: rs10821415, rs11196288, rs312949, rs1333042, rs1867624, rs2292318, rs2519093, rs35419456, rs7916879; and
AF-related single nucleotide polymorphism sites: rs11191416, rs1200159, rs12042319, rs2200733.

3. The method according to claim 1, wherein the individual information further comprises clinical factors, including the presence or absence of a stroke family history, hypertension, diabetes, dyslipidaemia and/or obesity.

4. The method according to claim 1, wherein a genetic risk score is obtained based on the information of respective single nucleotide polymorphism (SNP) sites in accordance with the following calculation:
Genetic risk score=Σβi×Ni
where βi is the effect value of the i^{th} SNP and Ni is the number of effect alleles of the i^{th} SNP carried by the individual;
preferably, the effect values of each SNP are shown in Table 3;
further preferably, the higher the genetic risk score, the higher the risk of stroke incidence in the individual;
even further preferably, said individual is from an East Asian population.

5. A device for evaluating a risk of stroke incidence comprising a detection unit and a data analysis unit, wherein:
the detection unit is used for detecting information from an individual to be evaluated and obtaining detection results; wherein the individual information is the individual information as defined in claim 1;
the data analysis unit is used for analyzing and processing the detection results from the detection unit;
preferably, the stroke comprises a haemorrhagic stroke and/or an ischaemic stroke.

6. The device for evaluating a risk of stroke incidence according to claim 5,
wherein the analyzing and processing the detection results from the detection unit by the data analysis unit comprises: assigning weight factors to the detection results of the single nucleotide polymorphism (SNP) sites to calculate a genetic risk score of the individual to be evaluated;
preferably, the data analysis unit comprises:
a preprocessing module for normalizing the detection results of the single nucleotide polymorphism sites;
a calculation module for bringing the normalized detection results of the single nucleotide polymorphism sites into following evaluation model to obtain a genetic risk score for the individual to be evaluated:
Genetic risk score=Σβi×Ni
where βi is the effect value of the i^{th} SNP and Ni is the number of effect alleles of the i^{th} SNP carried by the individual.

7. The device for evaluating a risk of stroke incidence according to claim 6, wherein the calculation module is used to evaluate lifetime stroke risk information by further combining the genetic risk score with clinical factors.

8. The device for evaluating a risk of stroke incidence according to claim 6, wherein the data analysis unit further comprises:
a matrix input module for receiving a plurality of the normalized detection results output by the preprocessing module, and inputting the normalized detection results in a matrix form to the calculation module;
preferably, the data analysis unit further comprises:
an output module for receiving the genetic risk score and/or the lifetime stroke risk information output by the calculation module and outputting it as a diagnostic classification result.

9. The device for evaluating a risk of stroke incidence according to claim 6, wherein the device is a computer storage medium storing computer program instructions, wherein when the computer program instructions are executed, an evaluation result of the risk of stroke incidence in an individual is obtained based on the information of the individual to be evaluated.

10. The device for evaluating a risk of stroke incidence according to claim 6, wherein the device is a computer device comprising a memory, a processor, and a computer program that is stored in the memory and executable on the processor, wherein when the processor executes the computer program, an evaluation result of the risk of stroke incidence in an individual is obtained based on the information of the individual to be evaluated.
